(19) United States
(12) Patent Application Publication
(10) Pub. No.: US 2008/0096906 A1
(43) Pub. Date: Apr. 24, 2008

(54) AMINOMETHYL-2-IMIDAZOLES

(76) Inventors: Guido Galley, Rheinfelden (DE); Annick Goergler, Colmar (FR); Katrin Groebke Zbinden, Liestal (CH); Roger Norcross, Olsberg (CH); Henri Stalder, Basel (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 11/872,192

(22) Filed: Oct. 15, 2007

(30) Foreign Application Priority Data
Oct. 19, 2006 (EP) 06122553.8

(51) Int. Cl. A61K 31/506; A61K 31/4178; A61P 25/18; A61P 25/28; A61P 3/04; C07D 233/61; C07D 401/12; C07D 403/12; C07D 233/66; A61P 3/10; A61P 25/30; A61P 25/24; A61K 31/4439

(52) U.S. Cl. 514/272; 514/256; 514/341; 514/397; 514/400; 544/328; 544/331; 544/333; 546/272.7; 548/315.1; 548/335.5; 548/340.1

(57) ABSTRACT

The present invention relates to compounds of formula I 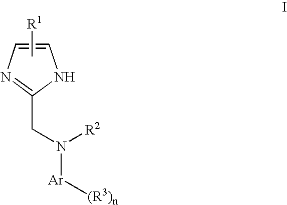 wherein
R¹ is selected from the group consisting of hydrogen or lower alkyl;
R² is hydrogen, lower alkyl, lower alkenyl, lower alkyl substituted by hydroxy, lower alkyl substituted by halogen,
   —(CH₂)ₓ—S-lower alkyl,
   —(CH₂)ₓ—O-lower alkyl,
   —(CH₂)ₓ—NHC(O)O-lower alkyl,
   —(CH₂)ₓ-aryl, and
   —(CH₂)ₓ-heteroaryl;
each R³ is selected from the group consisting of hydrogen, lower alkyl, lower alkoxy, halogen, hydroxy, lower alkyl substituted by halogen,
   —O—(CH₂)ₘ-aryl,
   —O—(CH₂)ₘ-heteroaryl,
   —(CR₂)ₘ-aryl, and
   —(CR₂)ₘ-heteroaryl; each R is selected from the group consisting of hydrogen, lower alkyl and hydroxy; Ar is selected from the group consisting of phenyl, pyrimidin-2-yl, pyrimidin-4-yl and pyridin-3-yl; n is 0, 1 or 2; x is 0, 1, 2 or 3; m is 0 or 1; and to their pharmaceutically active salts.

The invention relates also to processes for preparing such compounds, compositions comprising such a compound or a pharmaceutically-active salt thereof, and a method of treating a disease or disorder in a patient comprising administering such a compound, or pharmaceutically-active salt thereof, to a patient in need of such treatment.

### PRIORITY TO RELATED APPLICATION(S)

This application claims the benefit of European Patent Application No. 06122553.8, filed Oct. 19, 2006, which is hereby incorporated by reference in its entirety.

### FIELD OF THE INVENTION

This invention relates to compounds which have a good affinity to the trace amine associated receptors (TAARs), especially for TAAR1.

The invention relates also to processes for preparing such compounds, a pharmaceutical composition comprising such a compound, and a method for treating a disease or disorder in a patient comprising administering such a compound to a patient in need of such treatment.

### BACKGROUND OF THE INVENTION

It has been found that the compounds of formula I have a good affinity to the trace amine associated receptors (TAARs), especially for TAAR1.

The compounds may be used for the treatment of depression, anxiety disorders, bipolar disorder, attention deficit hyperactivity disorder (ADHD), stress-related disorders, psychotic disorders such as schizophrenia, neurological diseases such as Parkinson's disease, neurodegenerative disorders such as Alzheimer's disease, epilepsy, migraine, hypertension, substance abuse and metabolic disorders such as eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders.

The classical biogenic amines (serotonin, norepinephrine, epinephrine, dopamine, histamine) play important roles as neurotransmitters in the central and peripheral nervous system. Deutch, A. Y. and Roth, R. H. (1999) Neurotransmitters. In Fundamental Neuroscience (2^{nd} edn) (Zigmond, M. J., Bloom, F. E., Landis, S. C., Roberts, J. L, and Squire, L. R., eds.), pp. 193-234, Academic Press. Their synthesis and storage, as well as their degradation and reuptake after release are tightly regulated. An imbalance in the levels of biogenic amines is known to be responsible for the altered brain function under many pathological conditions. Wong, M. L. and Licinio, J. (2001) Nat. Rev. Neurosci. 2, 343-351; Carlsson, A. et al. (2001) Annu. Rev. Pharmacol. Toxicol. 41, 237-260; Tuite, P. and Riss, J. (2003) Expert Opin. Investig. Drugs 12, 1335-1352; Castellanos, F. X. and Tannock, R. (2002) Nat. Rev. Neurosci. 3, 617-628.

A second class of endogenous amine compounds, the so-called trace amines (TAs) significantly overlap with the classical biogenic amines regarding structure, metabolism and subcellular localization. The TAs include p-tyramine, β-phenylethylamine, tryptamine and octopamine, and they are present in the mammalian nervous system at generally lower levels than classical biogenic amines. Usdin, Earl; Sandler, Merton; Editors. Psychopharmacology Series, Vol. 1: Trace Amines and the Brain. [Proceedings of a Study Group at the 14th Annual Meeting of the American College of Neuropsychopharmacology, San Juan, Puerto Rico] (1976). Their disregulation has been linked to various psychiatric diseases like schizophrenia and depression and for other conditions like attention deficit hyperactivity disorder, migraine headache, Parkinson's disease, substance abuse and eating disorders. Lindemann, L. and Hoener, M. (2005) Trends in Pharmacol. Sci. 26, 274-281; Branchek, T. A. and Blackburn, T. P. (2003) Curr. Opin. Pharmacol. 3, 90-97; Premont, R. T. et al. (2001) Proc. Natl. Acad. Sci. U.S.A. 98, 9474-9475.

For a long time, TA-specific receptors had only been hypothesized based on anatomically discrete high-affinity TA binding sites in the central nervous system of humans and other mammals. Mousseau, D. D. and Butterworth, R. F. (1995) Prog. Brain Res. 106, 285-291; McCormack, J. K. et al. (1986) J. Neurosci. 6, 94-101. Accordingly, the pharmacological effects of TAs were believed to be mediated through the well known machinery of classical biogenic amines, by either triggering their release, inhibiting their reuptake or by “cross reacting” with their receptor systems. Premont, R. T. et al. (2001) Proc. Natl. Acad. Sci. U.S.A. 98, 9474-9475; Dyck, L. E. (1989) Life Sci. 44, 1149-1156; Parker, E. M. and Cubeddu, L. X. (1988) J. Pharmacol. Exp. Ther. 245, 199-210. This view changed significantly with the recent identification of several members of a novel family of GPCRs, the trace amine associated receptors (TAARs). Lindemann, L. and Hoener, M. (2005) Trends in Pharmacol. Sci. 26, 274-281; Lindemann, L. et al. (2005), Genomics 85, 372-385. There are 9 TAAR genes in human (including 3 pseudogenes) and 16 genes in mouse (including 1 pseudogene). The TAAR genes do not contain introns (with one exception, TAAR2 contains 1 intron) and are located next to each other on the same chromosomal segment. The phylogenetic relationship of the receptor genes, in agreement with an in-depth GPCR pharmacophore similarity comparison and pharmacological data suggest that these receptors form three distinct subfamilies. Lindemann, L. and Hoener, M. (2005) Trends in Pharmacol. Sci. 26, 274-281; Lindemann, L. et al. (2005), Genomics 85, 372-385. TAAR1 is in the first subclass of four genes (TAAR1-4) highly conserved between human and rodents. TAs activate TAAR1 via Gαs. Dysregulation of TAs was shown to contribute to the aetiology of various diseases like depression, psychosis, attention deficit hyperactivity disorder, substance abuse, Parkinson's disease, migraine headache, eating disorders, metabolic disorders and therefore TAAR1 ligands have a high potential for the treatment of these diseases.

The present invention relates to compounds which have a good affinity to the trace amine associated receptors (TAARs), especially for TAAR1.

### SUMMARY OF THE INVENTION

The present invention relates to a compound of formula I 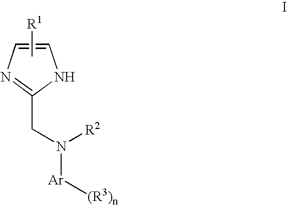
wherein
R¹ is selected from the group consisting of hydrogen and lower alkyl;
R² is selected from the group consisting of hydrogen, lower alkyl, lower alkenyl, lower alkyl substituted by hydroxy,
   lower alkyl substituted by halogen,
   —(CH₂)ₓ—S-lower alkyl,
   —(CH₂)ₓ—O-lower alkyl,
   —(CH₂)ₓ—NHC(O)O-lower alkyl,
   —(CH₂)ₓ-aryl, and
   —(CH₂)ₓ-heteroaryl;
each R³ is independently selected from the group consisting of hydrogen, lower alkyl, lower alkoxy, halogen, hydroxy, lower alkyl substituted by halogen,
   —O—(CH₂)ₘ-aryl,
   —O—(CH₂)ₘ-heteroaryl,
   —(CR₂)ₘ-aryl and
   —(CR₂)ₘ-heteroaryl;
each R is independently selected from the group consisting of hydrogen, lower alkyl and hydroxy;
Ar is selected from the group consisting of phenyl, pyrimidin-2-yl, pyrimidin-4-yl and pyridin-3-yl;
n is 0, 1 or 2;
x is 0, 1, 2 or 3; and
m is 0 or 1;
   and to a pharmaceutically-active salt of the above compound;
   with the proviso that the compound is not:
(1H-imidazol-2-ylmethyl)-phenyl-amine;
(4-fluoro-phenyl)-(1H-imidazol-2-ylmethyl)-amine;
(4-chloro-phenyl)-(1H-imidazol-2-ylmethyl)-amine;
(1H-imidazol-2-ylmethyl)-(2-methoxy-phenyl)-amine;
(3-chloro-phenyl)-(1H-imidazol-2-ylmethyl)-amine;
benzyl-(1H-imidazol-2-ylmethyl)-phenyl-amine;
ethyl-(1H-imidazol-2-ylmethyl)-phenyl-amine; or
(3,4-dichloro-phenyl)-(1H-imidazol-2-ylmethyl)-amine.

A further aspect of the present invention are processes for the preparation of the above compound.

Yet another aspect of the present invention is a pharmaceutical composition comprising the above compound or pharmaceutically-acceptable salt thereof.

Yet another aspect of the present invention is a method for treating a disease or disorder in a patient comprising administering the above compound, or pharmaceutically-acceptable salt thereof, to a patient in need of such treatment.

### DETAILED DESCRIPTION OF THE INVENTION

The present invention relates to a compound of formula I 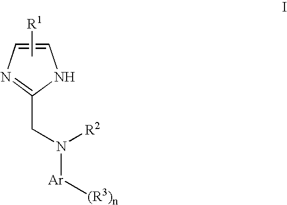
wherein
R¹ is selected from the group consisting of hydrogen and lower alkyl;
R² is selected from the group consisting of hydrogen, lower alkyl, lower alkenyl, lower alkyl substituted by hydroxy,
   lower alkyl substituted by halogen,
   —(CH₂)ₓ—S-lower alkyl,
   —(CH₂)ₓ—O-lower alkyl,
   —(CH₂)ₓ—NHC(O)O-lower alkyl,
   —(CH₂)ₓ-aryl, and
   —(CH₂)ₓ-heteroaryl;
each R³ is independently selected from the group consisting of hydrogen, lower alkyl, lower alkoxy, halogen, hydroxy, lower alkyl substituted by halogen,
   —O—(CH₂)ₘ-aryl,
   —O—(CH₂)ₘ-heteroaryl,
   —(CR₂)ₘ-aryl and
   —(CR₂)ₘ-heteroaryl;
each R³ is independently selected from the group consisting of hydrogen, lower alkyl and hydroxy;
Ar is selected from the group consisting of phenyl, pyrimidin-2-yl, pyrimidin-4-yl and pyridin-3-yl;
n is 0, 1 or 2;
x is 0, 1, 2 or 3; and
m is 0 or 1;
   and to a pharmaceutically-active salt of the above compound;
   with the proviso that the compound is not:
(1H-imidazol-2-ylmethyl)-phenyl-amine;
(4-fluoro-phenyl)-(1H-imidazol-2-ylmethyl)-amine;
(4-chloro-phenyl)-(1H-imidazol-2-ylmethyl)-amine;
(1H-imidazol-2-ylmethyl)-(2-methoxy-phenyl)-amine;
(3-chloro-phenyl)-(1H-imidazol-2-ylmethyl)-amine;
benzyl-(1H-imidazol-2-ylmethyl)-phenyl-amine;
ethyl-(1H-imidazol-2-ylmethyl)-phenyl-amine; or
(3,4-dichloro-phenyl)-(1H-imidazol-2-ylmethyl)-amine.

The invention includes all racemic mixtures, all their corresponding enantiomers and/or optical isomers. In addition, all tautomeric forms of compounds of formula I are also encompassed by the present invention.

Such compounds have a good affinity to the trace amine associated receptors (TAARs), especially for TAAR1 and may be used in the control or prevention of illnesses such as depression, anxiety disorders, bipolar disorder, attention deficit hyperactivity disorder, stress-related disorders, psychotic disorders such as schizophrenia, neurological diseases such as Parkinson's disease, neurodegenerative disorders such as Alzheimer's disease, epilepsy, migraine, hypertension, substance abuse and metabolic disorders such as eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders.

In preferred embodiments, the compounds of the present invention, or their pharmaceutically-acceptable salts, are used for treating depression, psychosis, Parkinson's disease, anxiety and attention deficit hyperactivity disorder (ADHD).

As used herein, the term “lower alkyl” denotes a saturated straight- or branched-chain group containing from 1 to 7 carbon atoms, for example, methyl, ethyl, propyl, isopropyl, n-butyl, i-butyl, 2-butyl, t-butyl and the like. Preferred alkyl groups are groups with 1-4 carbon atoms.

As used herein, the term “lower alkenyl” denotes a straight- or branched-chain group containing from 2 to 7 carbon atoms, wherein at least one bond is a double bond.

As used herein, the term “lower alkoxy” denotes a substituent in which an alkyl group is attached via an oxygen atom to the remainder of the molecule.

As used herein, the term “lower alkyl substituted by halogen” denotes an alkyl group as defined above, wherein at least one hydrogen atom is replaced by halogen, for example CF₃, CHF₂, CH₂F, CH₂CF₃, CH₂CH₂CF₃, CH₂CF₂CF₃ and the like.

As used herein, the term “aryl” denotes an aromatic group, selected from the group consisting of phenyl, naphthalen-1-yl and naphthalen-2-yl.

As used herein, the term “heteroaryl” is an aromatic group, containing at least one O, N or S ring atom, selected from the group consisting of thiophenyl, pyridinyl, pyrimidinyl, benzofuranyl and indolyl.

The term “halogen” denotes chlorine, iodine, fluorine or bromine.

The term “pharmaceutically-active salt” embraces salts with inorganic and organic acids, such as hydrochloric acid, nitric acid, sulfuric acid, phosphoric acid, citric acid, formic acid, fumaric acid, maleic acid, acetic acid, succinic acid, tartaric acid, methane-sulfonic acid, p-toluenesulfonic acid and the like.

In an embodiment of the present invention, the compound is a compound according to formula IA 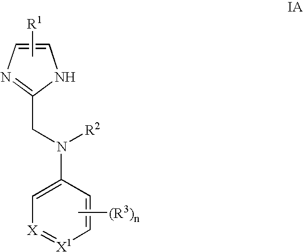
wherein
R¹ is selected from the group consisting of hydrogen and lower alkyl;
R² is selected from the group consisting of hydrogen, lower alkyl, lower alkenyl, lower alkyl substituted by hydroxy,
   lower alkyl substituted by halogen,
   —(CH₂)ₓ—S-lower alkyl,
   —(CH₂)ₓ—O-lower alkyl,
   —(CH₂)ₓ—NHC(O)O-lower alkyl,
   —(CH₂)ₓ-aryl and
   —(CH₂)ₓ-heteroaryl;
R³ is selected from the group consisting of hydrogen, lower alkyl, lower alkoxy, halogen, hydroxy, lower alkyl
   substituted by halogen,
   —O—(CH₂)ₘ-aryl,
   —O—(CH₂)ₘ-heteroaryl,
   —(CR₂)ₘ-aryl and
   —(CR₂)ₘ-heteroaryl;
each R is independently selected from the group consisting of hydrogen, lower alkyl and hydroxy;
X and X¹ are each independently selected from the group consisting of CH and N, with the provisio that X and X¹ are not both N;
n is 0, 1 or 2;
x is 0, 1, 2 or 3; and
m is 0 or 1;
   or a pharmaceutically active salt thereof; with the proviso that the compound is not:
(1H-imidazol-2-ylmethyl)-phenyl-amine;
(4-fluoro-phenyl)-(1H-imidazol-2-ylmethyl)-amine;
(4-chloro-phenyl)-(1H-imidazol-2-ylmethyl)-amine;
(1H-imidazol-2-ylmethyl)-(2-methoxy-phenyl)-amine;
(3-chloro-phenyl)-(1H-imidazol-2-ylmethyl)-amine;
benzyl-(1H-imidazol-2-ylmethyl)-phenyl-amine;
ethyl-(1H-imidazol-2-ylmethyl)-phenyl-amine; or
(3,4-dichloro-phenyl)-(1H-imidazol-2-ylmethyl)-amine.

Preferred compounds of the present invention are those of formula I wherein Ar is phenyl. Especially preferred from this group are those wherein R² is lower alkyl, for example:
(3-chloro-phenyl)-(1H-imidazol-2-ylmethyl)-methyl-amine;
(4-chloro-phenyl)-(1H-imidazol-2-ylmethyl)-methyl-amine;
(1H-imidazol-2-ylmethyl)-(3-methoxy-phenyl)-methyl-amine;
(4-fluoro-phenyl)-(1H-imidazol-2-ylmethyl)-isopropyl-amine;
ethyl-(3-fluoro-phenyl)-(1H-imidazol-2-ylmethyl)-amine;
(3-chloro-phenyl)-(1H-imidazol-2-ylmethyl)-isopropyl-amine;
(3-chloro-phenyl)-ethyl-(1H-imidazol-2-ylmethyl)-amine;
(2,5-difluoro-phenyl)-(3H-imidazol-4-ylmethyl)-isopropyl-amine;
(1H-imidazol-2-ylmethyl)-isopropyl-m-tolyl-amine;
(3-benzyloxy-phenyl)-(1H-imidazol-2-ylmethyl)-isopropyl-amine;
(1H-imidazol-2-ylmethyl)-isopropyl-[3-(pyridin-3-yloxy)-phenyl]-amine;
(3-benzyl-phenyl)-(1H-imidazol-2-ylmethyl)-isopropyl-amine;
biphenyl-3-yl-(1H-imidazol-2-ylmethyl)-isopropyl-amine;
[3-(4-chloro-phenoxy)-phenyl]-(1H-imidazol-2-ylmethyl)-isopropyl-amine;
Ethyl-(1H-imidazol-2-ylmethyl)-(3-phenoxy-phenyl)-amine;
(3-benzyloxy-phenyl)-ethyl-(1H-imidazol-2-ylmethyl)-amine; and
(3,4-dichloro-phenyl)-ethyl-(1H-imidazol-2-ylmethyl)-amine.

Further preferred are compounds of formula I wherein Ar is phenyl and R² is —(CH₂)ₓ—O-lower alkyl, for example the following compound:
(1H-imidazol-2-ylmethyl)-(2-methoxy-ethyl)-phenyl-amine.

Further preferred are compounds of formula I wherein Ar is phenyl and R² is hydrogen, for example the following compounds:
(1H-imidazol-2-ylmethyl)-(3-phenoxy-phenyl)-amine;
(3-benzyloxy-phenyl)-(1H-imidazol-2-ylmethyl)-amine;
(4-chloro-3-methoxy-phenyl)-(1H-imidazol-2-ylmethyl)-amine;
(3-bromo-phenyl)-(1H-imidazol-2-ylmethyl)-amine;
(3,4-difluoro-phenyl)-(1H-imidazol-2-ylmethyl)-amine;
(3-chloro-4-fluoro-phenyl)-(1H-imidazol-2-ylmethyl)-amine; and
(3,4-dichloro-phenyl)-(1H-imidazol-2-ylmethyl)-amine.

A further embodiment of the invention are compounds or formula I wherein Ar is selected from the group consisting of: pyrimidin-2-yl, pyrimidin-4-yl, and pyridin-3-yl.

The present compounds of formula I and their pharmaceutically-acceptable salts can be prepared by methods known in the art, for example, by processes described below.

One such process comprises reacting a compound of formula II 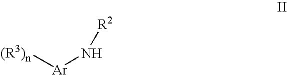
with a compound of formula III 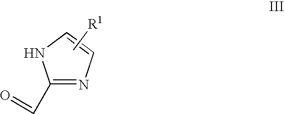
to produce a compound of formula I 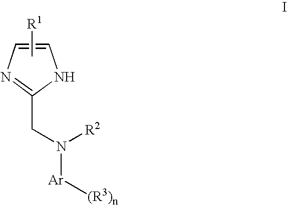
wherein R¹, R², R³, n and Ar are as defined above.

Another such process comprises reacting a compound of formula I-1 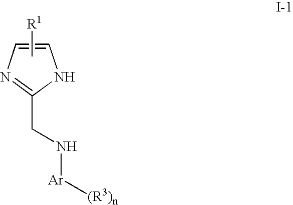
with a compound of formula R²—CHO
to produce a compound of formula I-2 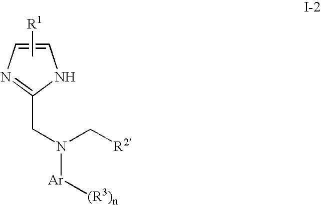
wherein R² is selected from the group consisting of lower alkyl, lower alkenyl, lower alkyl substituted by hydroxy, lower alkyl substituted by halogen, —(CH₂)ₓ₋₁—S-lower alkyl, —(CH₂)ₓ₋₁—O-lower alkyl, (CH₂)ₓ₋₁—NHC(O)O-lower alkyl and (CH₂)ₓ₋₁-heteroaryl; and the other substituents are as defined above.

Yet another such process comprises reacting a compound of formula I-1 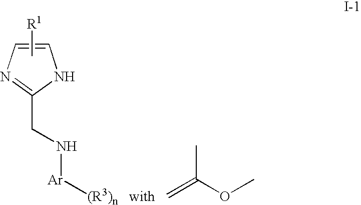
to produce a compound of formula I-3 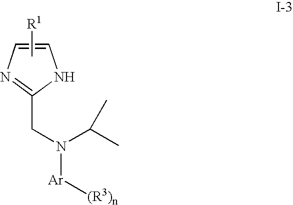
wherein the substituents are as defined above.

Yet another such process comprises removing a protecting group (denoted as “PG” below) from a compound of formula V or a compound of formula VIII. Preferred protecting groups are benzyl (on O) and 2-trimethylsilanyl-ethoxymethyl (on N). 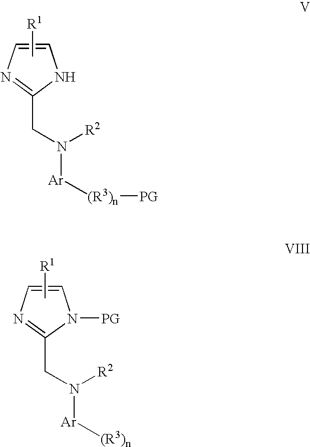
to produce a compound of formula I 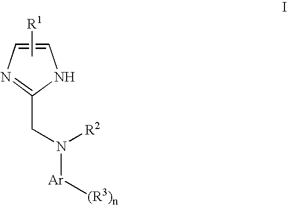
wherein the substituents are as defined above.

Yet another such process comprises reducing a compound of formula VII 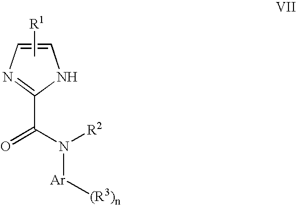
to a compound of formula VIII

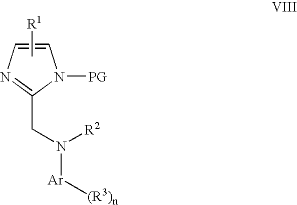

and removing the protecting group (PG) to produce a compound of formula I, wherein the substituents are as defined above. A preferred protecting groups is 2-trimethylsilanyl-ethoxymethyl (on N).

Yet another such process comprises reacting a compound of formula XII 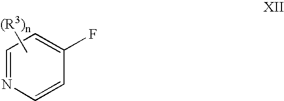
with a compound of formula XI 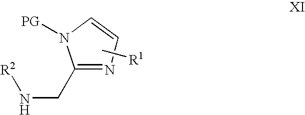
to produce a compound of formula XIII 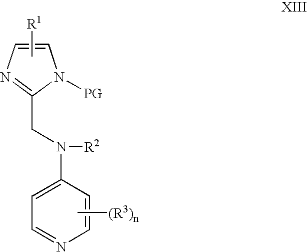
and removing the protecting group (PG) to produce a compound of formula I-4

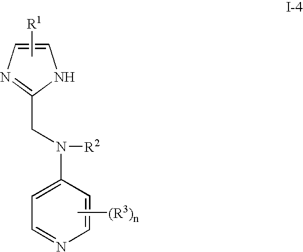

wherein the substituents are as defined above. A preferred protecting groups is 2-trimethylsilanyl-ethoxymethyl (on N).

If desired, the compound obtained by one of the processes described above may be converted into a pharmaceutically-acceptable salt.

The following are general schemes which exemplify the use of the above processes in the production of compounds of formula I. The starting materials are either commercially available, (e.g. from one or more of the following chemical suppliers such as Aldrich, Fluka, Acros, Maybridge, Avocado, TCI, or additional suppliers as indicated in databases such as Chemical Abstracts [American Chemical Society, Columbuis, Ohio] or Available Chemicals Directory [Elsevier MDL, San Ramon, Calif.])”, are otherwise known in the chemical literature, or may be prepared in accordance with methods described in the specific examples.

### Method 1

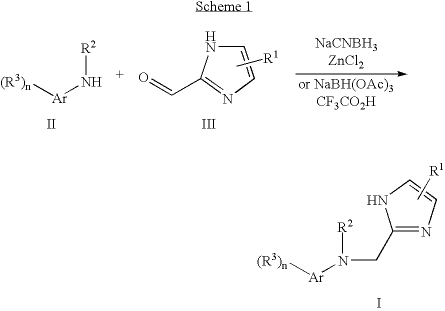

Compounds of formula I may be prepared by reductive amination using an aniline of formula II and an imidazole-2-carbaldehyde of formula III in the presence of NaCNBH₃ or NaBH(OAc)₃.

### Method 2

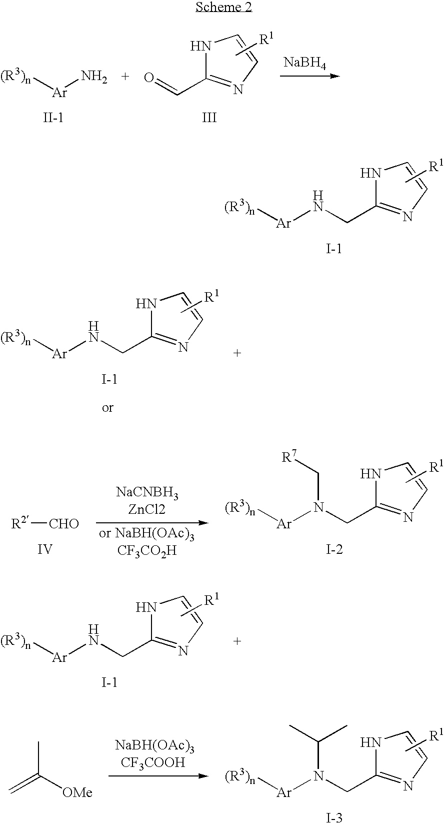 wherein R² is selected from the group consisting of lower alkyl, lower alkenyl, lower alkyl substituted by hydroxy, lower alkyl substituted by halogen, —(CH₂)ₓ₋₁—S-lower alkyl, —(CH₂)ₓ₋₁—O-lower alkyl, (CH₂)ₓ₋₁—NHC(O)O-lower alkyl and (CH₂)ₓ₋₁-heteroaryl.

Scheme 2 describes the preparation of compounds of formula I-1, I-2 or 1-3 by reductive amination followed by N-derivatization.

### Method 3

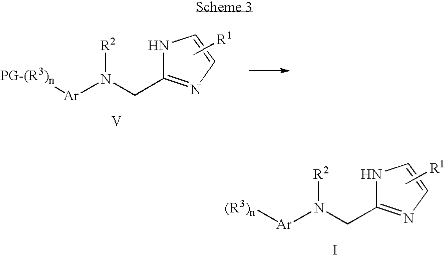

Scheme 3 describes the deprotection of a compound of formula V to produce a compound of formula I. The deprotection is carried out in usual matter. Compounds of formula V may be prepared according to methods 1 or 2. “PG” refers to a protecting group.

### Method 4

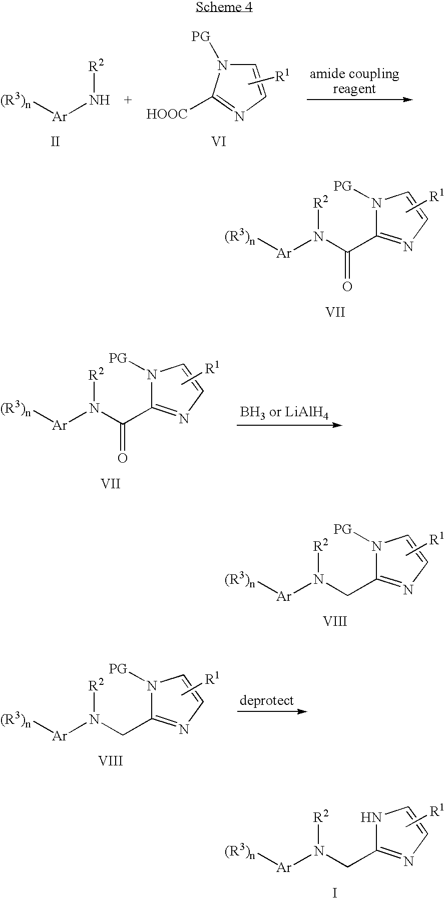

Scheme 4 describes the preparation of a compound of formula I by formation of an amide followed by reduction of the amide bond and protecting group removal. “PG” refers to a protecting group.

### Method 5

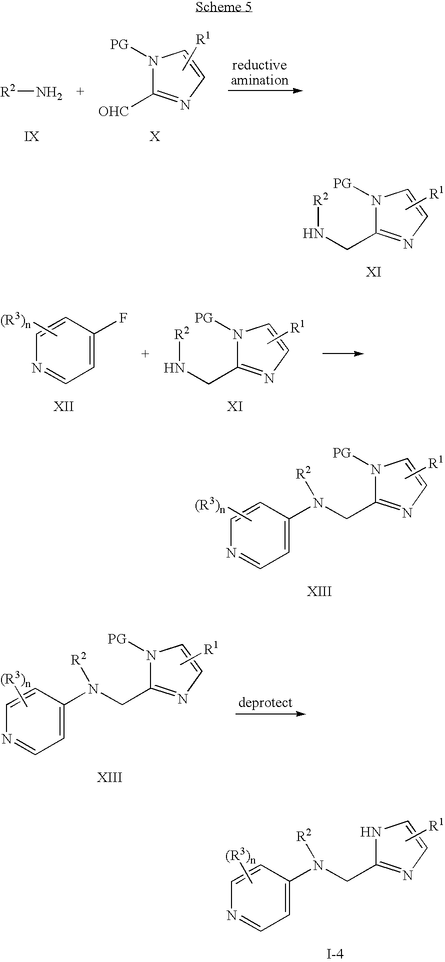

Scheme 5 describes the preparation of a compound of formula I-4 (X¹ is N) by formation of pyridine compounds by reaction of a 4-fluoropyridine to a protected aminomethylimidazole. PG” refers to a protecting group.

Isolation and Purification of the Compounds

Isolation and purification of the compounds and intermediates described herein can be effected, if desired, by any suitable separation or purification procedure such as, for example, filtration, extraction, crystallization, column chromatography, thin-layer chromatography, thick-layer chromatography, preparative low or high-pressure liquid chromatography or a combination of these procedures. Specific illustrations of suitable separation and isolation procedures can be had by reference to the preparations and examples herein below. However, other equivalent separation or isolation procedures could, of course, also be used. Racemic mixtures of chiral compounds of formula I can be separated using chiral HPLC.

Salts of Compounds of Formula I

The compounds of formula I are basic and may be converted to a corresponding acid addition salt. The conversion is accomplished by treatment with at least a stoichiometric amount of an appropriate acid, such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like. Typically, the free base is dissolved in an inert organic solvent such as diethyl ether, ethyl acetate, chloroform, ethanol or methanol and the like, and the acid added in a similar solvent. The temperature is maintained between 0° C. and 50° C. The resulting salt precipitates spontaneously or may be brought out of solution with a less polar solvent.

The acid addition salts of the basic compounds of formula I may be converted to the corresponding free bases by treatment with at least a stoichiometric equivalent of a suitable base such as sodium or potassium hydroxide, potassium carbonate, sodium bicarbonate, ammonia, and the like.

The compounds of formula I and their pharmaceutically-acceptable salts possess valuable pharmacological properties. Specifically, it has been found that the compounds of the present invention have a good affinity to the trace amine associated receptors (TAARs), especially TAAR1.

Compositions containing a compound of formula I or a pharmaceutically-acceptable salt thereof and a therapeutically inert carrier are also an object of the present invention, as is a process for their production, which comprises bringing one or more compounds of formula I and/or pharmaceutically acceptable salts thereof and, if desired, one or more other therapeutically valuable substances into a galenical administration form together with one or more therapeutically inert carriers.

The pharmaceutical compositions can be administered orally, e.g. in the form of tablets, coated tablets, dragées, hard and soft gelatine capsules, solutions, emulsions or suspensions. The administration can also be effected rectally, e.g. in the form of suppositories, and parenterally, e.g. in the form of injection solutions.

The compounds of formula I can be processed with pharmaceutically inert, inorganic or organic carriers for the production of pharmaceutical compositions. Lactose, corn starch or derivatives thereof, talc, stearic acids or its salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance, no carriers are usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically-valuable substances.

The present invention relates also to a method for treating a disease or disorder in a patient comprising administering a therapeutically-effective amount of a compound of the present invention to a patient in need of such treatment. A “therapeutically-effective amount” is the amount of the subject compound that will elicit the biological or medical response of a tissue, system, animal or human that is being sought by the researcher, veterinarian, medical doctor or other clinician. The above method may involve the administration of a composition which comprises a therapeutically-effective amount of the compound such as the compositions described above.

In preferred embodiments, the compound is used to treat disorders of the central nervous system, for example the treatment or prevention of schizophrenia, depression, cognitive impairment and Alzheimer's disease.

The therapeutically-effective amount can vary within wide limits and will, of course, have to be adjusted to the individual requirements in each particular case. In the case of oral administration the dosage for adults can vary from about 0.01 mg to about 1000 mg per day of a compound of general formula I or of the corresponding amount of a pharmaceutically acceptable salt thereof. The daily dosage may be administered as single dose or in divided doses and, in addition, the upper limit can also be exceeded when this is found to be indicated.

### Tablet Formulation (Wet Granulation)

| | | mg/tablet | | | |
|---|---|---|---|---|---|
| Item | Ingredients | 5 mg | 25 mg | 100 mg | 500 mg |
| 1. | Compound of formula I | 5 | 25 | 100 | 500 |
| 2. | Lactose Anhydrous DTG | 125 | 105 | 30 | 150 |
| 3. | Sta-Rx 1500 | 6 | 6 | 6 | 30 |
| 4. | Macrocrystalline Cellulose | 30 | 30 | 30 | 150 |
| 5. | Magnesium Stearate | 1 | 1 | 1 | 1 |
| | Total | 167 | 167 | 167 | 831 |

Manufacturing Procedure
1. Mix items 1, 2, 3 and 4 and granulate with purified water.
2. Dry the granules at 50° C.
3. Pass the granules through suitable milling equipment.
4. Add item 5 and mix for three minutes; compress on a suitable press.

### Capsule Formulation

| | | mg/capsule | | | |
|---|---|---|---|---|---|
| Item | Ingredients | 5 mg | 25 mg | 100 mg | 500 mg |
| 1. | Compound of formula I | 5 | 25 | 100 | 500 |
| 2. | Hydrous Lactose | 159 | 123 | 148 | — |
| 3. | Corn Starch | 25 | 35 | 40 | 70 |
| 4. | Talc | 10 | 15 | 10 | 25 |
| 5. | Magnesium Stearate | 1 | 2 | 2 | 5 |
| | Total | 200 | 200 | 300 | 600 |

Manufacturing Procedure
1. Mix items 1, 2 and 3 in a suitable mixer for 30 minutes.
2. Add items 4 and 5 and mix for 3 minutes.
3. Fill into a suitable capsule.

### EXAMPLES

The following examples illustrate the invention but are not intended to limit its scope.

### Example 1

### (1H-Imidazol-2-ylmethyl)-isopropyl-phenyl-amine

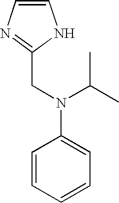

### a) (1H-Imidazol-2-ylmethyl)-phenyl-amine

To a solution of aniline (0.50 g, 5.37 mmol) in methanol (7 ml) was added imidazole-2-carboxyaldehyde (0.62 g, 6.45 mmol). After stirring the mixture overnight at room temperature sodium borohydride (0.305 g, 8.05 mmol) was added. The reaction mixture was stirred at room temperature for 4 hours. Then water was added and the mixture was extracted with ethyl acetate. The organic layer was separated, washed with water, dried over magnesium sulfate and evaporated. The residue was purified by crystallization (ethyl acetate) to yield a white solid (0.71 g, 76%); MS (ISP): 174.1 ((M+H)^{+.}).

### b) (1H-Imidazol-2-ylmethyl)-isopropyl-phenyl-amine

To a solution of (1H-imidazol-2-ylmethyl)-phenyl-amine (0.71 g, 4.1 mmol) in 1,2-dichloroethane (10 ml) were added successively 2-methoxypropene (0.5 ml, 5.3 mmol), trifluoroacetic acid (0.47 ml, 6.1 mmol) and sodium triacetoxyborohydride (1.3 g, 6.1 mmol). After stirring the mixture overnight at room temperature water and ethyl acetate were added. After extracting the aqueous phase twice with ethyl acetate the combined organic layers were dried over magnesium sulfate and evaporated. The residue was purified by column chromatography (SiO2, heptane/ethyl acetate=1:1) to yield a white solid (0.335 mg, 38%); MS (ISP): 216.2 ((M+H)^{+.}).

### Example 2

### (3-Chloro-phenyl)-(1H-imidazol-2-ylmethyl)-methyl-amine

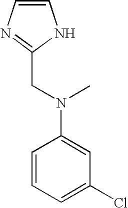

To a solution of 3-chloro-N-methylaniline (0.283 g, 2 mmol) in 1,2-dichloroethane (10 ml) were added molecular sieves (2 g, size 0.4 nM) and imidazol-2-carboxyaldehyde (0.288 g, 3 mmol). After stirring the mixture for 5 min at room temperature sodium triacetoxyborohydride (0.848 g, 4 mmol) and acetic acid (5 drops) were added. The reaction mixture was stirred at room temperature overnight. For workup dichloromethane (50 ml) and 1M sodium bicarbonate solution (30 ml) were added and the mixture was shaken. The organic layer was separated, dried over magnesium sulfate and evaporated. The residue was purified using flash chromatography (column: Isolute® Flash-NH₂ (Separtis); eluent: ethyl acetate/methanol=95:5) to yield a white solid (0.13 g, 29%); MS (ISP): 222.1 ((M+H)^{+.}).

### Example 3

### 2-[(1H-Imidazol-2-ylmethyl)-phenyl-amino]-ethanol

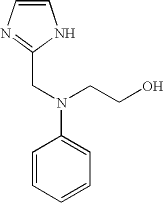

Analogously to Example 2, the title compound, MS (ISP): 218.2 ((M+H)^{+.}) was obtained in comparable yield using N-(2-hydroxyethyl)-aniline instead of 3-chloro-N-methylaniline.

### Example 4

### (1H-Imidazol-2-ylmethyl)-(4-methoxy-phenyl)-methyl-amine

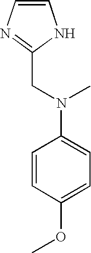

Analogously to Example 2, the title compound, MS (EI): 218.4 (M⁺) was obtained in comparable yield using N-methyl-4-methoxyaniline instead of 3-chloro-N-methylaniline.

### Example 5

### (4-Chloro-phenyl)-(1H-imidazol-2-ylmethyl)-methyl-amine

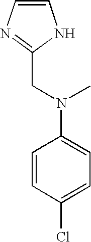

Analogously to Example 2, the title compound, MS (ISP): 222.1 ((M+H)^{+.}) was obtained in comparable yield using N-methyl-p-chloroaniline instead of 3-chloro-N-methylaniline.

### Example 6

### (1H-Imidazol-2-ylmethyl)-(3-methoxy-phenyl)-methyl-amine

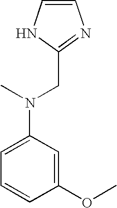

### a) 1-Benzyl-1H-imidazole-2-carboxylic acid (3-methoxy-phenyl)-methyl-amide

N-Methyl-3-methoxyaniline (0.302 g, 2.0 mmol) was dissolved in acetonitrile (10 ml). Then 1-benzyl-2-imidazolecarboxylic acid (0.302 g, 2.2 mmol), N-ethyldiisopropylamine (0.775 g, 6 mmol), 2-(1H-benzotriazol-1-yl)-1,1,3,3-tetramethyluronium tetrafluoroborate (TBTU; 1.0 g, 3.1 mmol) and for complete dissolution 2 ml of dimethylformamide were added. The reaction mixture was stirred overnight at room temperature. For workup, acetonitrile was evaporated in vacuo, sodium bicarbonate solution was added and the mixture was extracted twice with dichloromethane. The combined organic layers were dried over magnesium sulfate and evaporated. The residue was purified using chromatography (SiO₂; eluent: dichloromethane/methanol=97:3) to yield a light yellow oil (0.485 g, 75%); MS (ISP): 322.2 ((M+H)^{+.}).

### b) (1-Benzyl-1H-imidazol-2-ylmethyl)-(3-methoxy-phenyl)-methyl-amine

A solution of 1-benzyl-1H-imidazole-2-carboxylic acid (3-methoxy-phenyl)-methyl-amide (0.20 g, 0.64 mmol) was dissolved in tetrahydrofuran (5 ml). Then borane-tetrahydrofuran solution (3.6 ml, 1M, 3.6 mmol) was added at 0° C. and the reaction mixture was heated in a sealed tube for 4 hours. For workup hydrochloric acid (1M) was added until gas evolution stopped. Then the organic solvent was evaporated, more hydrochloric acid (3 ml, 1M) was added and the mixture was heated to 100° C. for 1 hour. After cooling, ammonium hydroxide solution (25%) was added until basic pH and the mixture was extracted with dichloromethane. The combined organic layers were dried over magnesium sulfate and evaporated. The residue was purified by flash chromatography (SiO2: heptane/ethyl acetate=1:1) to yield a light yellow oil (0.086 g, 44%); MS (ISP): 308.1 ((M+H)^{+.}).

### c) (1H-Imidazol-2-ylmethyl)-(3-methoxy-phenyl)-methyl-amine

Benzyl-1H-imidazol-2-ylmethyl)-(3-methoxy-phenyl)-methyl-amine (0.077 g, 0.25 mmol) was dissolved in ethanol (5 ml), acetic acid (0.075 g, 1.25 mmol) and palladium on charcoal (15 mg, 10% Pd) were added and the mixture was hydrogenated at 60° C. for 90 minutes. The catalyst was filtered off using Celite. To obtain the free base (remove acetic acid) the solution was put onto a SCX-column (0.5 g from Varian, sulfonic acid modified silica gel). After washing the SCX column with methanol (1 ml, discarded) the product was liberated from the column by washing with ammonia in methanol (2 ml, 1M). The solvent was evaporated and the residue was purified using column filtration (SiO2; dichloromethane/methanol=95:5) to yield an off-white solid (0.04 g, 74%); MS (ISP): 218.0 ((M+H)^{+.}).

### Example 7

### Isopropyl-(4-methyl-1H-imidazol-2-ylmethyl)-phenyl-amine

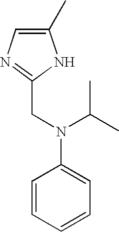

Analogously to Example 2, the title compound, MS (ISP): 230.1 ((M+H)^{+.}) was obtained in comparable yield using isopropyl-phenyl-amine instead of 3-chloro-N-methylaniline and 4-methyl-imidazol-2-carboxyaldehyde instead of imidazol-2-carboxyaldehyde.

### Example 8

### (3-Fluoro-phenyl)-(1H-imidazol-2-ylmethyl)-isopropyl-amine

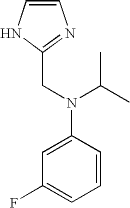

Analogously to Example 1, the title compound, MS (ISP): 234.3 ((M+H)^{+.}) was obtained in comparable yield using 3-fluoroaniline instead of aniline in step a).

### Example 9

### (2-Fluoro-phenyl)-(1H-imidazol-2-ylmethyl)-isopropyl-amine

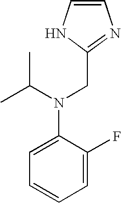

### a) 1-Benzyl-1H-imidazole-2-carboxylic acid (2-fluoro-phenyl)-isopropyl-amide

A mixture of 1-benzyl-2-imidazolecarboxylic acid (0.624 g, 3.0 mmol) and dichloromethylene-dimethyliminium chloride (0.487 g, 3.0 mmol) in dichloromethane (15 ml) was stirred at room temperature for 2 hours. Then N-isopropyl-2-fluoroaniline (0.306 g, 2.0 mmol) and sodium bicarbonate (0.840 g, 10 mmol) were added and stirring was continued overnight. For workup, water was added and the mixture was extracted twice with dichloromethane. The combined organic layers were dried over magnesium sulfate and evaporated. The residue was purified by chromatography (SiO₂; eluent: heptane/ethyl acetate=2:1) to yield a colorless oil (0.268 g, 40%); MS (ISP): 338.3 ((M+H)^{+.}).

### b) (2-Fluoro-phenyl)-(1H-imidazol-2-ylmethyl)-isopropyl-amine

The title compound, MS (ISP): 234.1 ((M+H)^{+.}) was obtained in comparable yield analogous to the procedure described for Example 6b) and c) using 1-benzyl-1H-imidazole-2-carboxylic acid (2-fluoro-phenyl)-isopropyl-amide instead of 1-benzyl-1H-imidazole-2-carboxylic acid (3-methoxy-phenyl)-methyl-amide in step b).

### Example 10

### (4-Fluoro-phenyl)-(1H-imidazol-2-ylmethyl)-isopropyl-amine

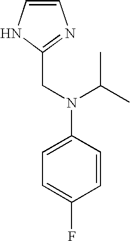

Analogously to Example 1, the title compound, MS (ISP): 234.0 ((M+H)^{+.}) was obtained in comparable yield using 4-fluoroaniline instead of aniline in step a).

### Example 11

### (1H-Imidazol-2-ylmethyl)-(2-isopropyl-6-methyl-phenyl)-amine

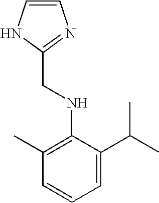

To a solution of 2-isopropyl-6-methyl-aniline (1.49 g, 10 mmol) in methanol (10 ml) was added imidazole-2-carboxyaldehyde (0.96 g, 10 mmol). After stirring the mixture overnight at 60° C., sodium borohydride (0.567 g, 15 mmol) was added. The reaction mixture was stirred at room temperature for 4 hours. Then water was added and the mixture was extracted with ethyl acetate. The organic layer was separated, washed with water, dried over magnesium sulfate and evaporated. The residue was purified by chromatography (column: Isolute® Flash-NH₂ (Separtis); eluent: heptane/ethyl acetate=1:1) to yield a white solid (1.29 g, 56%); MS (ISP): 230.1 ((M+H)^{+.}).

### Example 12

### Ethyl-(3-fluoro-phenyl)-(1H-imidazol-2-ylmethyl)-amine

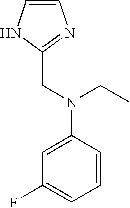

### a) (1H-Imidazol-2-ylmethyl)-(3-fluoro-phenyl)-amine

To a solution of 3-fluoroaniline (0.33 g, 3.0 mmol) in methanol (7 ml) was added imidazole-2-carboxyaldehyde (0.29 g, 3.0 mmol) and the mixture was stirred overnight at 60° C. After cooling, sodium borohydride (0.17 g, 4.5 mmol) was added and the reaction mixture was stirred at room temperature for 4 hours. Then water was added and the mixture was extracted with ethyl acetate. The organic layer was separated, washed with water, dried over magnesium sulfate and evaporated. The residue was purified by chromatography (SiO2, ethyl acetate) to yield a light yellow solid (0.315 g, 55%); MS (ISP): 192.1 ((M+H)^{+.}).

### b) Ethyl-(3-fluoro-phenyl)-(1H-imidazol-2-ylmethyl)-amine

(1H-Imidazol-2-ylmethyl)-(3-fluoro-phenyl)-amine (0.19 g, 1 mmol) was dissolved in methanol (15 ml). Then acetaldehyde (0.28 ml, 5 mmol), zinc chloride (0.55 g, 4 mmol) and sodium cyanoborohydride (0.31 g, 5 mmol) were added and the reaction mixture was allowed to stir at 40° C. overnight. After cooling, the reaction mixture was poured onto ammoniumchloride/ice and extracted with ethyl acetate (2 times 50 ml). The organic layer was dried over magnesium sulfate and evaporated. The residue was purified using flash chromatography (SiO₂; eluent: heptane/ethyl acetate=90:10) to yield an off-white solid (0.118 g, 54%); MS (ISP): 220.2 ((M+H)^{+.}).

### Example 13

### (2-Chloro-phenyl)-(1H-imidazol-2-ylmethyl)-amine

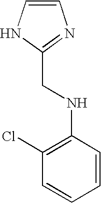

Analogously to Example 11, the title compound, MS (ISP): 208.1 ((M+H)^{+.}) was obtained in comparable yield using 2-chloroaniline instead of 2-isopropyl-6-methyl-aniline.

### Example 14

### (2-Chloro-phenyl)-(1H-imidazol-2-ylmethyl)-isopropyl-amine

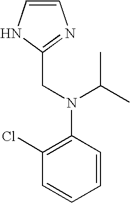

Analogously to Example 1, the title compound, MS (ISP): 250.1 ((M+H)^{+.}) was obtained in comparable yield using 2-chloroaniline instead of aniline in step a).

### Example 15

### (3-Chloro-phenyl)-(1H-imidazol-2-ylmethyl)-isopropyl-amine

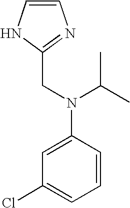

Analogously to Example 1, the title compound, MS (ISP): 250.3 ((M+H)^{+.}) was obtained in comparable yield using 3-chloroaniline instead of aniline in step a).

### Example 16

### (4-Chloro-phenyl)-(1H-imidazol-2-ylmethyl)-isopropyl-amine

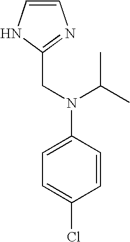

Analogously to Example 1, the title compound, MS (ISP): 250.1 ((M+H)^{+.}) was obtained in comparable yield using 4-chloroaniline instead of aniline in step a).

### Example 17

### (1H-Imidazol-2-ylmethyl)-isopropyl-(2-methoxy-phenyl)-amine

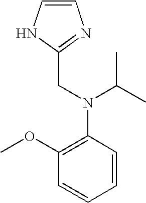

Analogously to Example 1, the title compound, MS (ISP): 246.4 ((M+H)^{+.}) was obtained in comparable yield using 2-methoxyaniline instead of aniline in step a).

### Example 18

### (1H-Imidazol-2-ylmethyl)-(3-methylsulfanyl-propyl)-phenyl-amine

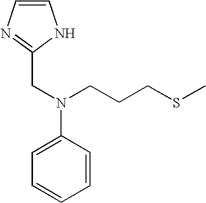

Analogously to Example 12, the title compound, MS (ISP): 262.1 ((M+H)^{+.}) was obtained in comparable yield using aniline instead of 3-fluoroaniline in step a) and 3-(methylthio)-propionaldehyde instead of acetaldehyde in step b).

### Example 19

### (1H-Imidazol-2-ylmethyl)-isobutyl-phenyl-amine

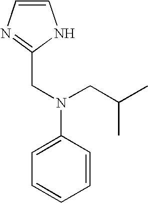

Analogously to Example 12, the title compound, MS (ISP): 230.3 ((M+H)^{+.}) was obtained in comparable yield using aniline instead of 3-fluoroaniline in step a) and isobutyraldehyde instead of acetaldehyde in step b).

### Example 20

### (1H-Imidazol-2-ylmethyl)-(3-methyl-but-2-enyl)-phenyl-amine; 1:1 mixture with (1H-imidazol-2-ylmethyl)-(3-methyl-butyl)-phenyl-amine

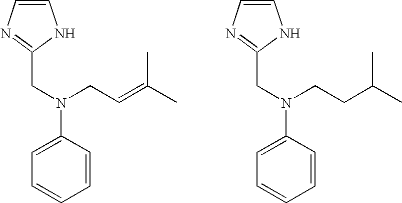

Analogously to Example 12, the title compound, MS (ISP): 242.1; 244.4 ((M+H)^{+.}) was obtained in comparable yield using aniline instead of 3-fluoroaniline in step a) and 3-methylcrotonaldehyde instead of acetaldehyde in step b).

### Example 21

### (1H-Imidazol-2-ylmethyl)-(2-methoxy-ethyl)-phenyl-amine

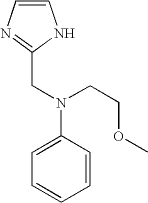

Analogously to Example 12, the title compound, MS (ISP): 232.3 ((M+H)^{+.}) was obtained in comparable yield using aniline instead of 3-fluoroaniline in step a) and methoxyacetaldehyde instead of acetaldehyde in step b).

### Example 22

### (1H-Imidazol-2-ylmethyl)-phenyl-(3,3,3-trifluoro-propyl)-amine

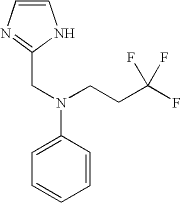

Analogously to Example 12, the title compound, MS (ISP): 270.4 ((M+H)^{+.}) was obtained using aniline instead of 3-fluoroaniline in step a) and 3,3,3-trifluoropropionaldehyde instead of acetaldehyde in step b).

### Example 23

### {2-[(1H-Imidazol-2-ylmethyl)-phenyl-amino]-ethyl}-carbamic acid tert-butyl ester

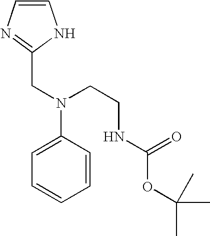

Analogously to Example 12, the title compound, MS (ISP): 317.4 ((M+H)^{+.}) was obtained in comparable yield using aniline instead of 3-fluoroaniline in step a) and tert-butyl N-(2-oxoethyl)carbamate instead of acetaldehyde in step b).

### Example 24

### (1H-Imidazol-2-ylmethyl)-phenyl-thiophen-3-ylmethyl-amine

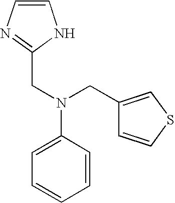

Analogously to Example 12, the title compound, MS (ISP): 270.3 ((M+H)^{+.}) was obtained in comparable yield using aniline instead of 3-fluoroaniline in step a) and 3-thiophenecarboxyaldehyde instead of acetaldehyde in step b).

### Example 25

### (1H-Imidazol-2-ylmethyl)-(3-methoxy-phenyl)-amine

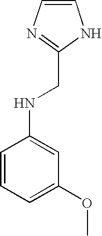

Analogously to Example 11, the title compound, MS (ISP): 203.9 ((M+H)^{+.}) was obtained in comparable yield using 3-methoxyaniline instead of 2-isopropyl-6-methyl-aniline.

### Example 26

### (2-Fluoro-phenyl)-(1H-imidazol-2-ylmethyl)-amine

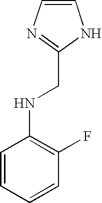

Analogously to Example 11, the title compound, MS (ISP): 192.0 ((M+H)^{+.}) was obtained in comparable yield using 2-fluoroaniline instead of 2-isopropyl-6-methyl-aniline.

### Example 27

### Ethyl-(1H-imidazol-2-ylmethyl)-(3-methoxy-phenyl)-amine

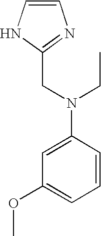

Analogously to Example 12, the title compound, MS (ISP): 231.9 ((M+H)^{+.}) was obtained in comparable yield using 3-methoxyaniline instead of 3-fluoroaniline in step a).

### Example 28

### Ethyl-(2-fluoro-phenyl)-(1H-imidazol-2-ylmethyl)-amine

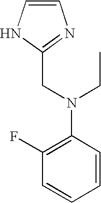

Analogously to Example 12, the title compound, MS (ISP): 220.1 ((M+H)^{+.}) was obtained in comparable yield using 2-fluoroaniline instead of 3-fluoroaniline in step a).

### Example 29

### Ethyl-(4-fluoro-phenyl)-(1H-imidazol-2-ylmethyl)-amine

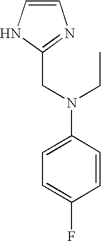

Analogously to Example 12, the title compound, MS (ISP): 220.2 ((M+H)^{+.}) was obtained in comparable yield using 4-fluoroaniline instead of 3-fluoroaniline in step a).

### Example 30

### (3-Chloro-phenyl)-ethyl-(1H-imidazol-2-ylmethyl)-amine

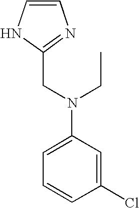

Analogously to Example 12, the title compound, MS (ISP): 235.9 ((M+H)^{+.}) was obtained in comparable yield using 3-chloroaniline instead of 3-fluoroaniline in step a).

### Example 31

### (1H-Imidazol-2-ylmethyl)-(2-methoxy-phenyl)-methyl-amine

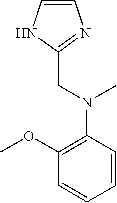

Analogously to Example 9, the title compound, MS (ISP): 218.4 ((M+H)^{+.}) was obtained in comparable yield using 2-methoxy-N-methylaniline instead of N-isopropyl-2-fluoroaniline in step a).

### Example 32

### (1H-Imidazol-2-ylmethyl)-methyl-phenyl-amine

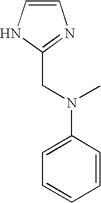

Analogously to Example 9, the title compound, MS (ISP): 188.3 ((M+H)^{+.}) was obtained in comparable yield using N-methylaniline instead of N-isopropyl-2-fluoroaniline in step a).

### Example 33

### (4-Fluoro-phenyl)-(1H-imidazol-2-ylmethyl)-methyl-amine

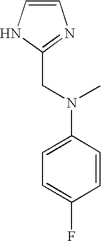

Analogously to Example 12, the title compound, MS (ISP): 206.1 ((M+H)^{+.}) was obtained in comparable yield using 4-fluoroaniline instead of 3-fluoroaniline in step a) and formaldehyde instead of acetaldehyde in step b).

### Example 34

### (6-Chloro-pyridin-3-yl)-ethyl-(1H-imidazol-2-ylmethyl)-amine

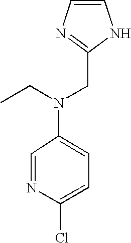

Analogously to Example 12, the title compound, MS (ISP): 208.6; 210.9 ((M+H)^{+.}) was obtained in comparable yield using 5-amino-2-chloropyridine instead of 3-fluoroaniline in step a).

### Example 35

### (1H-Imidazol-2-ylmethyl)-isopropyl-(3-methoxy-phenyl)-amine

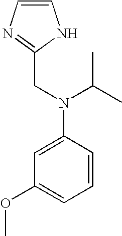

Analogously to Example 1, the title compound, MS (ISP): 246.3 ((M+H)^{+.}) was obtained in comparable yield using 3-methoxyaniline instead of aniline in step a).

### Example 36

### (1H-Imidazol-2-ylmethyl)-isopropyl-(3-trifluoromethoxy-phenyl)-amine

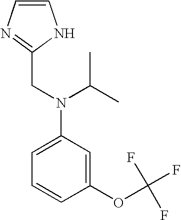

Analogously to Example 1, the title compound, MS (ISP): 300.1 ((M+H)^{+.}) was obtained using 3-trifluoromethoxy-aniline instead of aniline in step a).

### Example 37

### (2,5-Difluoro-phenyl)-(3H-imidazol-4-ylmethyl)-isopropyl-amine

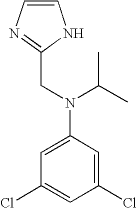

Analogously to Example 1, the title compound, MS (ISP): 284.2; 286.1 ((M+H)^{+.}) was obtained using 3,5-dichloroaniline instead of aniline in step a).

### Example 38

### (3-Chloro-5-fluoro-phenyl)-(1H-imidazol-2-ylmethyl)-isopropyl-amine

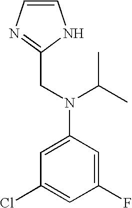

Analogously to Example 1, the title compound, MS (ISP): 268.2 ((M+H)^{+.}) was obtained in comparable yield using 3-chloro-5-fluoroaniline instead of aniline in step a).

### Example 39

### (1H-Imidazol-2-ylmethyl)-isopropyl-m-tolyl-amine

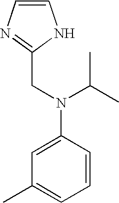

Analogously to Example 1, the title compound, MS (ISP): 230.3 ((M+H)^{+.}) was obtained in comparable yield using 3-methylaniline instead of aniline in step a).

### Example 40

### (4-Fluoro-3-methoxy-phenyl)-(1H-imidazol-2-ylmethyl)-isopropyl-amine

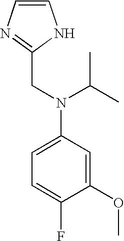

Analogously to Example 1, the title compound, MS (ISP): 264.0 ((M+H)^{+.}) was obtained in comparable yield using 4-fluoro-3-methoxyaniline instead of aniline in step a).

### Example 41

### (3-Benzyloxy-phenyl)-(1H-imidazol-2-ylmethyl)-isopropyl-amine

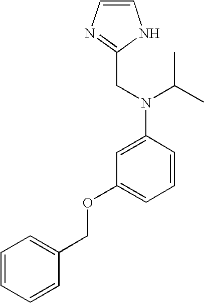

Analogously to Example 1, the title compound, MS (ISP): 322.3 ((M+H)^{+.}) was obtained in comparable yield using 3-benzyloxyaniline instead of aniline in step a).

### Example 42

### (3-Chloro-5-trifluoromethyl-phenyl)-(1H-imidazol-2-ylmethyl)-isopropyl-amine

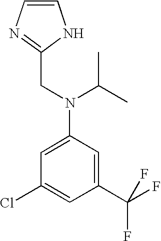

Analogously to Example 1, the title compound, MS (ISP): 318.1 ((M+H)^{+.}) was obtained in comparable yield using 3-chloro-5-trifluoromethyl-aniline instead of aniline in step a).

### Example 43

### (1H-Imidazol-2-ylmethyl)-isopropyl-[3-(pyridin-3-yloxy)-phenyl]-amine

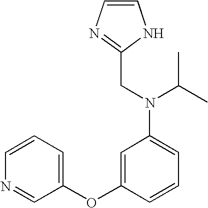

Analogously to Example 1, the title compound, MS (ISP): 308.4 ((M+H)^{+.}) was obtained in comparable yield using 3-(pyridin-3-yloxy)-phenyl-amine instead of aniline in step a).

### Example 44

### (1H-Imidazol-2-ylmethyl)-isopropyl-(3-phenoxy-phenyl)-amine

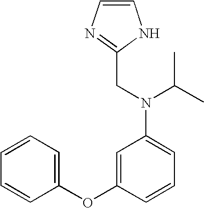

Analogously to Example 1, the title compound, MS (ISP): 309.3 ((M+H)^{+.}) was obtained in comparable yield using 3-phenoxy-aniline instead of aniline in step a).

### Example 45

### (3-Benzyl-phenyl)-(1H-imidazol-2-ylmethyl)-isopropyl-amine

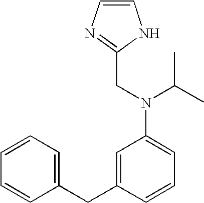

Analogously to Example 1, the title compound, MS (ISP): 306.5 ((M+H)^{+.}) was obtained in comparable yield using 3-benzylaniline instead of aniline in step a).

### Example 46

### Biphenyl-3-yl-(1H-imidazol-2-ylmethyl)-isopropyl-amine

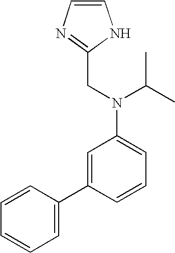

Analogously to Example 1, the title compound, MS (ISP): 292.3 ((M+H)^{+.}) was obtained in comparable yield using biphenyl-3-ylamine instead of aniline in step a).

### Example 47

### Biphenyl-3-yl-(1H-imidazol-2-ylmethyl)-isopropyl-amine

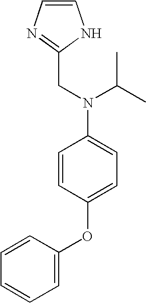

Analogously to Example 1, the title compound, MS (ISP): 308.5 ((M+H)^{+.}) was obtained in comparable yield using 4-phenoxy-aniline instead of aniline in step a).

### Example 48

### [4-(3,4-Dichloro-phenoxy)-phenyl]-(1H-imidazol-2-ylmethyl)-isopropyl-amine

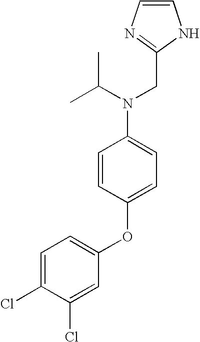

Analogously to Example 1, the title compound, MS (ISP): 376.1; 378.2 ((M+H)^{+.}) was obtained in comparable yield using 4-(3′,4′-dichlorophenoxy)-aniline instead of aniline in step a).

### Example 49

### (1H-Imidazol-2-ylmethyl)-isopropyl-[4-(4-trifluoromethyl-phenoxy)-phenyl]-amine

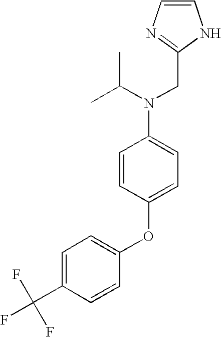

Analogously to Example 1, the title compound, MS (ISP): 376.2 ((M+H)^{+.}) was obtained in comparable yield using 4-(4-trifluoromethyl-phenoxy)-aniline instead of aniline in step a).

### Example 50

### (1H-Imidazol-2-ylmethyl)-isopropyl-[4-(4-methoxy-phenoxy)-phenyl]-amine

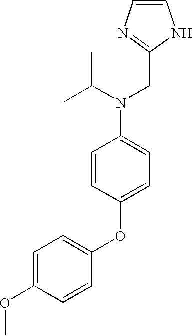

Analogously to Example 1, the title compound, MS (ISP): 338.2 ((M+H)^{+.}) was obtained in comparable yield using 4-(4-methoxy-phenoxy)-aniline instead of aniline in step a).

### Example 51

### [3-(4-Chloro-phenoxy)-phenyl]-(1H-imidazol-2-ylmethyl)-isopropyl-amine

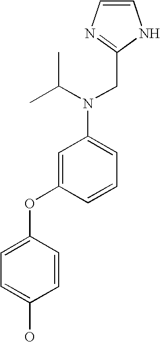

Analogously to Example 1, the title compound, MS (ISP): 342.1; 344.1 ((M+H)^{+.}) was obtained in comparable yield using 4-(4-chloro-phenoxy)-aniline instead of aniline in step a).

### Example 52

### (2,6-Difluoro-pyridin-4-yl)-ethyl-(1H-imidazol-2-ylmethyl)-amine

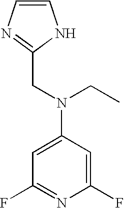

### a) Ethyl-[1-(2-trimethylsilanyl-ethoxymethyl)-1H-imidazol-2-ylmethyl]-amine

To a saturated solution of ethylamine in methanol (3 ml) was added 1-(2-trimethylsilyl)ethoxymethyl-2-imidazolecarboxaldehyde (0.2 g, 0.88 mmol) and the mixture was stirred for 1 hour. Sodium borohydride (0.05 g, 1.3 mmol) was added and the mixture was stirred overnight at 50° C.

Water was added and the solution was extracted three times with ethyl acetate. The combined organic layers were dried over magnesium sulfate and evaporated. The residue was purified by flash chromatography (SiO2: ethyl acetate/methanol=9:1) to yield a yellow oil (0.176 g, 78%); MS (ISP): 256.0 ((M+H)^{+.}).

### b) (2,6-Difluoro-pyridin-4-yl)-ethyl-(1H-imidazol-2-ylmethyl)-amine

A mixture of ethyl-[1-(2-trimethylsilanyl-ethoxymethyl)-1H-imidazol-2-ylmethyl]-amine (0.1 g, 0.39 mmol) and 2,4,6-trifluoropyridine (0.2 g; 1.5 mmol) was heated in a sealed vessel in a microwave oven for 1.5 h at 170° C. Then water and dichloromethane was added and the organic layer was separated, dried over magnesium sulfate and evaporated. To the residue tetrabutylammonium fluoride solution in tetrahydrofuran (1M, 1 ml, 1 mmol) was added and the mixture was stirred overnight. The solvent was evaporated and the residue was purified by flash chromatography (column: Isolute® Flash-NH₂ from Separtis; eluent: heptane/ethyl acetate=1:1) to yield a white solid, (0.01 g, 10%); MS (ISP): 239.0 ((M+H)^{+.}).

### Example 53

### {3-[(H-Imidazol-2-ylmethyl)-isopropyl-amino]-phenyl}-phenyl-methanol

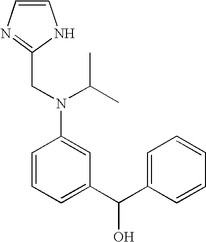

Analogously to Example 1, the title compound, MS (ISP): 322.4 ((M+H)^{+.}) was obtained in comparable yield using 3-aminobenzophenone instead of aniline in step a).

### Example 54

### 3-[(1H-Imidazol-2-ylmethyl)-isopropyl-amino]-phenol

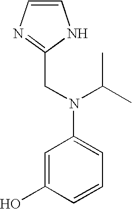

(3-Benzyloxy-phenyl)-(3H-imidazol-2-ylmethyl)-isopropyl-amine (0.285 g, 0.89 mmol) was dissolved in ethanol (5 ml), palladium on charcoal (30 mg, 10% Pd) was added and the mixture was hydrogenated for 5 hours at room temperature. The catalyst was filtered off and the solvent was evaporated. The residue was purified by flash chromatography (column: Isolute® Flash-NH₂ from Separtis; eluent: ethyl acetate) to yield a white foam, (0.162 g, 79%); MS (ISP): 232.1 ((M+H)^{+.}).

### Example 55

### (1H-Imidazol-2-ylmethyl)-isopropyl-[3-(pyridin-4-yloxy)-phenyl]-amine

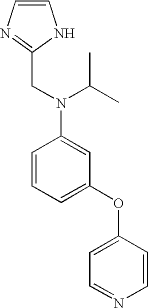

Analogously to Example 1, the title compound, MS (ISP): 309.3 ((M+H)^{+.}) was obtained in comparable yield using 3-(pyridin-4-yloxy)-phenyl-amine instead of aniline in step a).

### Example 56

### (1H-Imidazol-2-ylmethyl)-isopropyl-(3-pyrimidin-5-yl-phenyl)-amine

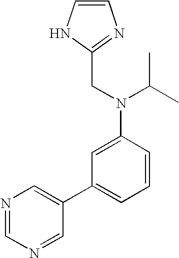

Analogously to Example 1, the title compound, MS (ISP): 294.0 ((M+H)^{+.}) was obtained in comparable yield using (3-pyrimidin-5-yl-phenyl)-amine instead of aniline in step a).

### Example 57

### Ethyl-(1H-imidazol-2-ylmethyl)-(4-methoxy-pyrimidin-2-yl)-amine

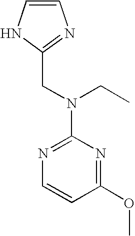

Analogously to Example 52, the title compound, MS (ISP): 234.1 ((M+H)^{+.}) was obtained in comparable yield using 2-chloro-4-methoxypyrimidine instead of 2,4,6-trifluoropyridine in step b).

### Example 58

### (2-Benzyl-6-chloro-pyrimidin-4-yl)-ethyl-(1H-imidazol-2-ylmethyl)-amine

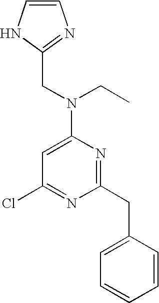

Analogously to Example 52, the title compound, MS (ISP): 328.2 ((M+H)^{+.}) was obtained in comparable yield using 2-benzyl-4,6-dichloropyrimidine instead of 2,4,6-trifluoropyridine in step b).

### Example 59

### (2-Benzyl-pyrimidin-4-yl)-ethyl-(1H-imidazol-2-ylmethyl)-amine

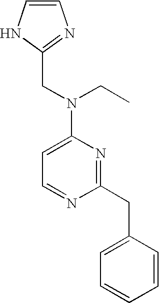

(2-Benzyl-6-chloro-pyrimidin-4-yl)-ethyl-(1H-imidazol-2-ylmethyl)-amine (0.164 g, 0.5 mmol) was dissolved in methanol (5 ml), ammonium formate (0.315 g, 0.5 mmol) and palladium on charcoal (0.164 g, 10% Pd) was added and the mixture was refluxed for one hour. After cooling the catalyst was filtered off and the solvent was evaporated. The residue was purified by flash chromatography (column: Isolute® Flash-NH₂ from Separtis; eluent: ethyl acetate/methanol=95:5) to yield a white solid, (0.100 g, 68%); MS (ISP): 294.4 ((M+H)^{+.}).

### Example 60

### (3,4-Dichloro-phenyl)-(1H-imidazol-2-ylmethyl)-isopropyl-amine

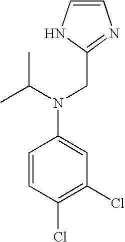

### a) (3,4-Dichloro-phenyl)-isopropyl-amine

3,4-Dichloroaniline (5.0 g, 30.86 mmol) was dissolved in methanol (150 ml). Then acetone (22.7 ml, 308.6 mmol), zinc chloride (12.62 g, 92.58 mmol) and sodium cyanoborohydride (7.76 g, 123.4 mmol) were added and the reaction mixture was allowed to stir at 40° C. overnight. After cooling, the reaction mixture was poured onto ammoniumchloride/ice and extracted with ethyl acetate (2 times 200 ml). The organic layer was dried over magnesium sulfate and evaporated. The residue was purified using flash chromatography (SiO₂; eluent: heptane/ethyl acetate=95:5) to yield an off-white solid (5.03 g, 79.9%); MS (ISP): 205.1 ([³⁷Cl M+H]^{+.}), 203.1 ([³⁵Cl M+H]⁺).

### b) (3,4-Dichloro-phenyl)-(1H-imidazol-2-ylmethyl)-isopropyl-amine

(3,4-Dichloro-phenyl)-isopropyl-amine (0.30 g, 1.47 mmol) was dissolved in methanol (10 ml). Then imidazole-2-carboxaldehyde (0.22 g, 2.20 mmol), zinc chloride (0.60 g, 4.4 mmol) and sodium cyanoborohydride (0.19 g, 2.9 mmol) were added and the reaction mixture was allowed to stir at 60° C. overnight. After cooling, the reaction mixture was concentrated in vacuo and the residue was purified using flash chromatography (SiO₂; eluent: dichloromethane/methanol gradient) to yield a white solid (0.007 g, 2%); MS (ISP): 286.0 ([³⁷Cl M+H]^{+.}), 284.0 ([³⁵Cl M+H]⁺).

### Example 61

### (1H-Imidazol-2-ylmethyl)-(3-phenoxy-phenyl)-amine

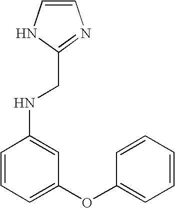

Analogously to Example 11, the title compound, MS (ISP): 266.2 ([M+H]⁺) was obtained in comparable yield using 3-phenoxyaniline instead of 2-isopropyl-6-methyl-aniline.

### Example 62

### (3-Benzyloxy-phenyl)-(1H-imidazol-2-ylmethyl)-amine

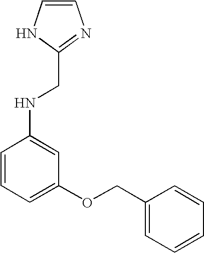

Analogously to Example 11, the title compound, MS (ISP): 280.2 ([M+H]⁺) was obtained in comparable yield using 3-benzyloxyaniline instead of 2-isopropyl-6-methyl-aniline.

### Example 63

### (4-Benzyloxy-phenyl)-(1H-imidazol-2-ylmethyl)-amine

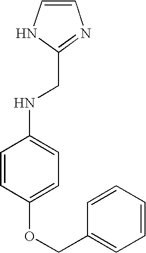

Analogously to Example 11, the title compound, MS (ISP): 280.0 ([M+H]⁺) was obtained in comparable yield using 4-benzyloxyaniline instead of 2-isopropyl-6-methyl-aniline.

### Example 64

### (4-Chloro-3-methoxy-phenyl)-(1H-imidazol-2-ylmethyl)-amine

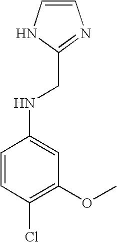

Analogously to Example 11, the title compound, MS (ISP): 240.2 ([³⁷Cl M+H]^{+.}), 238.0 ([³⁵Cl M+H]^{+.}) was obtained in comparable yield using 4-chloro-3-methoxyaniline instead of 2-isopropyl-6-methyl-aniline.

### Example 65

### Ethyl-(1H-imidazol-2-ylmethyl)-(3-phenoxy-phenyl)-amine

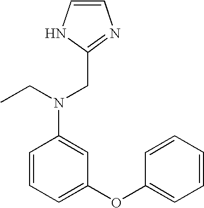

Analogously to Example 12, the title compound, MS (ISP): 294.2 ((M+H)^{+.}) was obtained in comparable yield using (1H-imidazol-2-ylmethyl)-(3-phenoxy-phenyl)-amine (Example 61) instead of (1H-imidazol-2-ylmethyl)-(3-fluoro-phenyl)-amine in step b).

### Example 66

### (3-Benzyloxy-phenyl)-ethyl-(1H-imidazol-2-ylmethyl)-amine

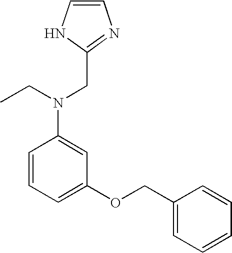

Analogously to Example 12, the title compound, MS (ISP): 308.4 ((M+H)^{+.}) was obtained in comparable yield using (3-benzyloxy-phenyl)-(1H-imidazol-2-ylmethyl)-amine (Example 62) instead of (1H-imidazol-2-ylmethyl)-(3-fluoro-phenyl)-amine in step b).

### Example 67

### (4-Benzyloxy-phenyl)-ethyl-(1H-imidazol-2-ylmethyl)-amine

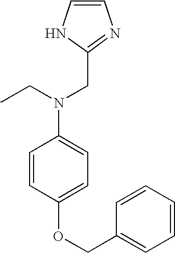

Analogously to Example 12, the title compound, MS (ISP): 308.3 ((M+H)^{+.}) was obtained in comparable yield using (4-benzyloxy-phenyl)-(1H-imidazol-2-ylmethyl)-amine (Example 63) instead of (1H-imidazol-2-ylmethyl)-(3-fluoro-phenyl)-amine in step b).

### Example 68

### (3,4-Dichloro-phenyl)-ethyl-(1H-imidazol-2-ylmethyl)-amine

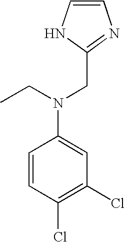

Analogously to Example 12, the title compound, MS (ISP): 272.1 ([³⁷Cl M+H]^{+.}), 270.2 ([³⁵Cl M+H]^{+.}) was obtained in comparable yield using 3,4-dichloroaniline in place of 3-fluoroaniline in step a) and using (3,4-dichloro-phenyl)-(1H-imidazol-2-ylmethyl)-amine instead of (1H-imidazol-2-ylmethyl)-(3-fluoro-phenyl)-amine in step b).

### Example 69

### (4-Chloro-3-methoxy-phenyl)-(1H-imidazol-2-ylmethyl)-methyl-amine

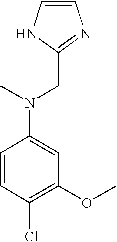

(4-Chloro-3-methoxy-phenyl)-(1H-imidazol-2-ylmethyl)-amine (0.10 g, 0.4 mmol) (Example 64) was dissolved in acetonitrile (4 ml). Then formaldehyde (0.08 ml, 1.1 mmol, 37% aq solution) and sodium cyanoborohydride (0.08 g, 1.3 mmol) were added and the reaction mixture was allowed to stir at room temperature overnight. The reaction mixture was concentrated in vacuo and the residue was purified using flash chromatography (SiO₂; eluent: methanol/dichloromethane gradient) to yield a white solid (0.006 g, 6%); MS (ISP): 252.1 ([³⁷Cl M+H]^{+.}), 250.2 ([³⁵Cl M+H]^{+.}).

### Example 70

### (4-Chloro-3-methoxy-phenyl)-(1H-imidazol-2-ylmethyl)-isopropyl-amine

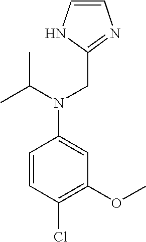

Analogously to Example 1, the title compound, MS (ISP): 282.1 ([³⁷Cl M+H]^{+.}), 280.2 ([³⁵Cl M+H]^{+.}). using (4-chloro-3-methoxy-phenyl)-(1H-imidazol-2-ylmethyl)-amine (Example 64) instead of (1H-imidazol-2-ylmethyl)-phenyl-amine in step b).

### Example 71

### (3-Bromo-phenyl)-(1H-imidazol-2-ylmethyl)-amine

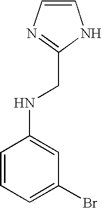

Analogously to Example 11, the title compound, MS (ISP): 251.9; 254.1 ((M+H)^{+.}) was obtained in comparable yield using 3-bromoaniline instead of 2-isopropyl-6-methyl-aniline.

### Example 72

### (1H-Imidazol-2-ylmethyl)-(4-methoxy-phenyl)-amine

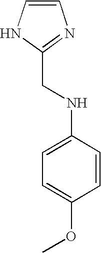

Analogously to Example 11, the title compound, MS (ISP): 203.9 ((M+H)^{+.}) was obtained in comparable yield using 4-methoxyaniline instead of 2-isopropyl-6-methyl-aniline.

### Example 73

### (3,4-Difluoro-phenyl)-(1H-imidazol-2-ylmethyl)-amine

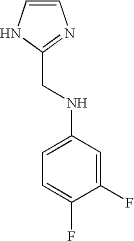

Analogously to Example 11, the title compound, MS (ISP): 209.9 ((M+H)^{+.}) was obtained in comparable yield using 3,4-difluoroaniline instead of 2-isopropyl-6-methyl-aniline.

### Example 74

### (3-Chloro-4-fluoro-phenyl)-(1H-imidazol-2-ylmethyl)-amine

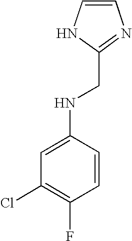

Analogously to Example 11, the title compound, MS (ISP): 228.1 ([³⁷Cl M+H]^{+.}), 226.1 ([³⁵Cl M+H]^{+.}) was obtained in comparable yield using 3-chloro-4-fluoroaniline instead of 2-isopropyl-6-methyl-aniline.

### Example 75

### (3-Bromo-phenyl)-(1H-imidazol-2-ylmethyl)-isopropyl-amine

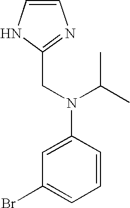

Analogously to Example 1, the title compound, MS (ISP): 296.1; 294.1 ((M+H)^{+.}) was obtained in comparable yield using 3-bromoaniline instead of aniline in step a).

### Example 76

### (1H-Imidazol-2-ylmethyl)-isopropyl-(4-methoxy-phenyl)-amine

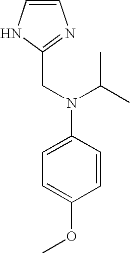

Analogously to Example 1, the title compound, MS (ISP): 246.2 ((M+H)^{+.}) was obtained in comparable yield using 4-methoxyaniline instead of aniline in step a).

### Example 77

### (3,4-Difluoro-phenyl)-(1H-imidazol-2-ylmethyl)-isopropyl-amine

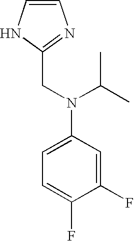

Analogously to Example 1, the title compound, MS (ISP): 252.1 ((M+H)^{+.}) was obtained in comparable yield using 3,4-difluoroaniline instead of aniline in step a).

### Example 78

### (3-Chloro-4-fluoro-phenyl)-(1H-imidazol-2-ylmethyl)-isopropyl-amine

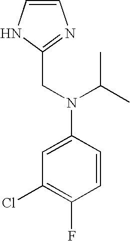

Analogously to Example 1, the title compound, MS (ISP): 268.1; 270.1 ((M+H)^{+.}) was obtained in comparable yield using 3-chloro-4-fluoroaniline instead of aniline in step a).

### Example 79

### (3-Bromo-phenyl)-ethyl-(1H-imidazol-2-ylmethyl)-amine

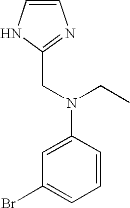

Analogously to Example 1, the title compound, MS (ISP): 279.9; 281.9 ((M+H)^{+.}) was obtained in comparable yield using 3-bromoaniline instead of aniline in step a).

### Example 80

### Ethyl-(1H-imidazol-2-ylmethyl)-(4-methoxy-phenyl)-amine

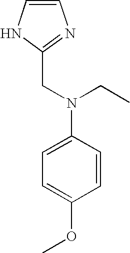

Analogously to Example 12, the title compound, MS (ISP): 231.9 ((M+H)^{+.}) was obtained in comparable yield using 4-methoxyaniline instead of 3-fluoroaniline in step a).

### Example 81

### (3,4-Difluoro-phenyl)-ethyl-(1H-imidazol-2-ylmethyl)-amine

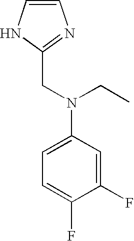

Analogously to Example 12, the title compound, MS (ISP): 237.9 ((M+H)^{+.}) was obtained in comparable yield using 3,4-difluoroaniline instead of 3-fluoroaniline in step a).

### Example 82

### (3-Chloro-4-fluoro-phenyl)-ethyl-(1H-imidazol-2-ylmethyl)-amine

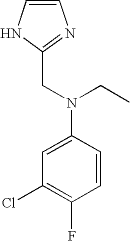

Analogously to Example 12, the title compound, MS (ISP): 253.9; 255.8 ((M+H)^{+.}) was obtained in comparable yield using 3-chloro-4-fluoroaniline instead of 3-fluoroaniline in step a).

### Example 83

### 2-[Biphenyl-3-yl-(1H-imidazol-2-ylmethyl)-amino]-ethanol

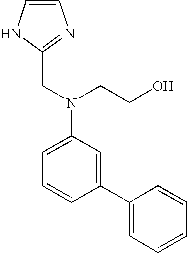

Analogously to Example 2, the title compound, MS (ISP): 294.1 ((M+H)^{+.}) was obtained in comparable yield using 2-(biphenyl-3-ylamino)-ethanol instead of 3-chloro-N-methylaniline.

### Example 84

The ability of the compounds of the present invention to bind to TAAR1 was investigated in accordance with the test given hereinafter.

### Materials and Methods

Construction of TAAR Expression Plasmids and Stably Transfected Cell Lines

For the construction of expression plasmids the coding sequences of human, rat and mouse TAAR 1 were amplified from genomic DNA essentially as described by Lindemann et al. (2005) Genomics 85, 372-385. The Expand High Fidelity PCR System (Roche Diagnostics) was used with 1.5 mM Mg²⁺ and purified PCR products were cloned into pCR2.1-TOPO cloning vector (Invitrogen) following the instructions of the manufacturer. PCR products were subcloned into the pIRESneo2 vector (BD Clontech, Palo Alto, Calif.), and expression vectors were sequence verified before introduction in cell lines.

HEK293 cells (ATCC # CRL-1573) were cultured essentially as described Lindemann et al. (2005) Genomics 85, 372-385. For the generation of stably transfected cell lines HEK293 cells were transfected with the pIRESneo2 expression plasmids containing the TAAR coding sequences (described above) with Lipofectamine 2000 (Invitrogen) according to the instructions of the manufacturer, and 24 hours post transfection the culture medium was supplemented with 1 mg/ml G418 (Sigma, Buchs, Switzerland). After a culture period of about 10 days, clones were isolated, expanded and tested for responsiveness to trace amines (all compounds purchased from Sigma) with the cAMP Biotrak Enzyme immunoassay (EIA) System (Amersham) following the non-acetylation EIA procedure provided by the manufacturer. Monoclonal cell lines which displayed a stable EC₅₀ for a culture period of 15 passages were used for all subsequent studies.

Membrane Preparation and Radioligand Binding

Cells at confluence were rinsed with ice-cold phosphate buffered saline without Ca²⁺ and Mg²⁺ containing 10 mM EDTA and pelleted by centrifugation at 1000 rpm for 5 min at 4° C. The pellet was then washed twice with ice-cold phosphate buffered saline and cell pellet was frozen immediately by immersion in liquid nitrogen and stored until use at −80° C. The cell pellet was then suspended in 20 ml HEPES-NaOH (20 mM), pH 7.4 containing 10 mM EDTA, and homogenized with a Polytron (PT 3000, Kinematica) at 10,000 rpm for 10 seconds. The homogenate was centrifuged at 48,000×g for 30 minutes at 4° C. and the pellet resuspended in 20 ml HEPES-NaOH (20 mM), pH 7.4 containing 0.1 mM EDTA (buffer A), and homogenized with a Polytron at 10,000 rpm for 10 seconds. The homogenate was then centrifuged at 48,000×g for 30 minutes at 4° C. and the pellet resuspended in 20 ml buffer A, and homogenized with a Polytron at 10,000 rpm for 10 seconds. Protein concentration was determined by the method of Pierce (Rockford, Ill.). The homogenate was then centrifuged at 48,000×g for 10 minutes at 4° C., resuspended in HEPES-NaOH (20 mM), pH 7.0 including MgCl₂ (10 mM) and CaCl₂ (2 ml) (buffer B) at 200 μg protein per ml and homogenized with a Polytron at 10,000 rpm for 10 seconds.

Binding assay was performed at 4° C. in a final volume of 1 ml, and with an incubation time of 30 minutes. The radioligand [³H]-rac-2-(1,2,3,4-tetrahydro-1-naphthyl)-2-imidazoline was used at a concentration equal to the calculated K_{d} value of 60 nM to give a total binding at around 0.1% of the total added radioligand concentration, and a specific binding which represented approximately 70-80% of the total binding. Non-specific binding was defined as the amount of [³H]-rac-2-(1,2,3,4-tetrahydro-1-naphthyl)-2-imidazoline bound in the presence of the appropriate unlabelled ligand (10 μM). Competing ligands were tested in a wide range of concentrations (10 pM-30 μM). The final dimethylsulphoxide concentration in the assay was 2%, and it did not affect radioligand binding. Each experiment was performed in duplicate. All incubations were terminated by rapid filtration through UniFilter-96 plates (Packard Instrument Company) and glass filter GF/C, pre-soaked for at least 2 h in polyethylenimine 0.3%, and using a Filtermate 96 Cell Harvester (Packard Instrument Company). The tubes and filters were then washed 3 times with 1 ml aliquots of cold buffer B. Filters were not dried and soaked in Ultima gold (45 μl/well, Packard Instrument Company) and bound radioactivity was counted by a TopCount Microplate Scintillation Counter (Packard Instrument Company).

The preferred compounds show a Ki value (μM) in mouse on TAAR1 in the range of 0.002-0.100 as shown in the table below.

| | Ki (μM) | | |
|---|---|---|---|
| Example | mouse | Example | Ki |
| 2 | 0.030 | 46 | 0.077 |
| 5 | 0.081 | 51 | 0.082 |
| 6 | 0.073 | 61 | 0.0261 |
| 10 | 0.094 | 62 | 0.0028 |
| 12 | 0.051 | 64 | 0.0259 |
| 15 | 0.050 | 65 | 0.0111 |
| 21 | 0.082 | 66 | 0.0263 |
| 30 | 0.036 | 68 | 0.0331 |
| 37 | 0.065 | 71 | 0.0995 |
| 39 | 0.089 | 73 | 0.0708 |
| 41 | 0.021 | 74 | 0.0839 |
| 43 | 0.025 | K8 | 0.055 |
| 45 | 0.002 | | |

K8=(3,4-dichloro-phenyl)-(1H-imidazol-2-ylmethyl)-amine.

1. A compound according to formula I 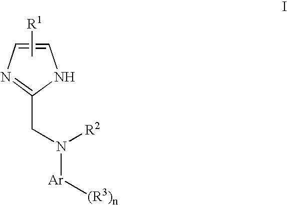
wherein
R¹ is selected from the group consisting of: hydrogen and lower alkyl;
R² is selected from the group consisting of: hydrogen, lower alkyl, lower alkenyl, lower alkyl substituted by hydroxy,
lower alkyl substituted by halogen,
—(CH₂)ₓ—S-lower alkyl,
—(CH₂)ₓ—O-lower alkyl,
—(CH₂)ₓ—NHC(O)O-lower alkyl,
—(CH₂)ₓ-aryl and
—(CH₂)ₓ-heteroaryl;
each R³ is independently selected from the group consisting of: hydrogen, lower alkyl, lower alkoxy, halogen, hydroxy, lower alkyl substituted by halogen,
—O—(CH₂)ₘ-aryl,
—O—(CH₂)ₘ-heteroaryl,
—(CR₂)ₘ-aryl and
—(CR₂)ₘ-heteroaryl;
each R is independently selected from the group consisting of: hydrogen, lower alkyl and hydroxy;
Ar is selected from the group consisting of: phenyl, pyrimidin-2-yl, pyrimidin-4-yl and pyridin-3-yl;
n is 0, 1 or 2;
x is 0, 1, 2 or 3; and
m is 0 or 1;
or a pharmaceutically active salt thereof,
with the proviso that the compound is not:
(1H-imidazol-2-ylmethyl)-phenyl-amine;
(4-fluoro-phenyl)-(1H-imidazol-2-ylmethyl)-amine;
(4-chloro-phenyl)-(1H-imidazol-2-ylmethyl)-amine;
(1H-imidazol-2-ylmethyl)-(2-methoxy-phenyl)-amine;
(3-chloro-phenyl)-(1H-imidazol-2-ylmethyl)-amine;
benzyl-(1H-imidazol-2-ylmethyl)-phenyl-amine;
ethyl-(1H-imidazol-2-ylmethyl)-phenyl-amine; and
(3,4-dichloro-phenyl)-(1H-imidazol-2-ylmethyl)-amine.

2. A compound according to claim 1 wherein said compound is a compound according to formula IA: 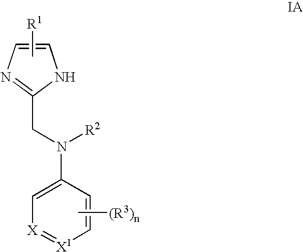
wherein
X and X¹ are each independently selected from the group consisting of CH and N, with the provisio that X and X¹ are not both N;
or a pharmaceutically active salt thereof;
with the proviso that the compound is not:
(1H-imidazol-2-ylmethyl)-phenyl-amine;
(4-fluoro-phenyl)-(1H-imidazol-2-ylmethyl)-amine;
(4-chloro-phenyl)-(1H-imidazol-2-ylmethyl)-amine;
(1H-imidazol-2-ylmethyl)-(2-methoxy-phenyl)-amine;
(3-chloro-phenyl)-(1H-imidazol-2-ylmethyl)-amine;
benzyl-(1H-imidazol-2-ylmethyl)-phenyl-amine;
ethyl-(1H-imidazol-2-ylmethyl)-phenyl-amine; and
(3,4-dichloro-phenyl)-(1H-imidazol-2-ylmethyl)-amine.

3. A compound according to claim 1 wherein Ar is phenyl.

4. A compound according to claim 3 wherein R² is lower alkyl.

5. A compound according to claim 4 selected from the group consisting of:
(3-chloro-phenyl)-(1H-imidazol-2-ylmethyl)-methyl-amine;
(4-chloro-phenyl)-(1H-imidazol-2-ylmethyl)-methyl-amine;
(1H-imidazol-2-ylmethyl)-(3-methoxy-phenyl)-methyl-amine;
(4-fluoro-phenyl)-(1H-imidazol-2-ylmethyl)-isopropyl-amine;
ethyl-(3-fluoro-phenyl)-(1H-imidazol-2-ylmethyl)-amine;
(3-chloro-phenyl)-(1H-imidazol-2-ylmethyl)-isopropyl-amine;
(3-chloro-phenyl)-ethyl-(1H-imidazol-2-ylmethyl)-amine;
(2,5-difluoro-phenyl)-(3H-imidazol-4-ylmethyl)-isopropyl-amine;
(1H-imidazol-2-ylmethyl)-isopropyl-m-tolyl-amine;
(3-benzyloxy-phenyl)-(1H-imidazol-2-ylmethyl)-isopropyl-amine;
(1H-imidazol-2-ylmethyl)-isopropyl-[3-(pyridin-3-yloxy)-phenyl]-amine;
(3-benzyl-phenyl)-(1H-imidazol-2-ylmethyl)-isopropyl-amine;
biphenyl-3-yl-(1H-imidazol-2-ylmethyl)-isopropyl-amine;
[3-(4-chloro-phenoxy)-phenyl]-(1H-imidazol-2-ylmethyl)-isopropyl-amine;
Ethyl-(1H-imidazol-2-ylmethyl)-(3-phenoxy-phenyl)-amine;
(3-benzyloxy-phenyl)-ethyl-(1H-imidazol-2-ylmethyl)-amine; and
(3,4-dichloro-phenyl)-ethyl-(1H-imidazol-2-ylmethyl)-amine.

6. A compound according to claim 3 wherein R² is —(CH₂)ₓ—O-lower alkyl.

7. A compound according to claim 6 wherein said compound is (1H-imidazol-2-ylmethyl)-(2-methoxy-ethyl)-phenyl-amine.

8. A compound according to claim 3 wherein R² is hydrogen.

9. A compound according to claim 8 selected from the group consisting of:
(1H-imidazol-2-ylmethyl)-(3-phenoxy-phenyl)-amine;
(3-benzyloxy-phenyl)-(1H-imidazol-2-ylmethyl)-amine;
(4-chloro-3-methoxy-phenyl)-(1H-imidazol-2-ylmethyl)-amine;
(3-bromo-phenyl)-(1H-imidazol-2-ylmethyl)-amine;
(3,4-difluoro-phenyl)-(1H-imidazol-2-ylmethyl)-amine;
(3-chloro-4-fluoro-phenyl)-(1H-imidazol-2-ylmethyl)-amine; and
(3,4-dichloro-phenyl)-(1H-imidazol-2-ylmethyl)-amine.

10. A compound according to claim 1 wherein Ar is pyrimidin-2-yl.

11. A compound according to claim 1 wherein Ar is pyrimidin-4-yl.

12. A compound according to claim 1 wherein Ar is pyridin-3-yl.

13. A process for the preparation of a compound according to formula I 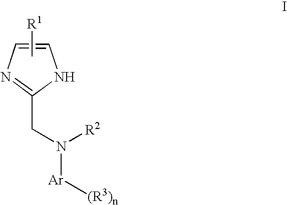
, said process comprising reacting a compound of formula II 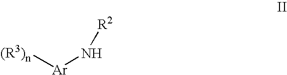
with a compound of formula III 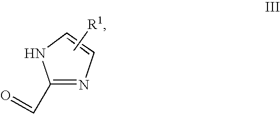
wherein, in the above formulas,
R¹ is selected from the group consisting of hydrogen and lower alkyl;
R² is selected from the group consisting of: hydrogen, lower alkyl, lower alkenyl, lower alkyl substituted by hydroxy,
lower alkyl substituted by halogen,
—(CH₂)ₓ—S-lower alkyl,
—(CH₂)ₓ—O-lower alkyl,
—(CH₂)ₓ—NHC(O)O-lower alkyl,
—(CH₂)ₓ-aryl and
—(CH₂)ₓ-heteroaryl;
each R³ is independently selected from the group consisting of: hydrogen, lower alkyl, lower alkoxy, halogen, hydroxy, lower alkyl substituted by halogen,
—O—(CH₂)ₘ-aryl,
—O—(CH₂)ₘ-heteroaryl,
—(CR₂)ₘ-aryl and
—(CR₂)ₘ-heteroaryl;
each R is independently selected from the group consisting of: hydrogen, lower alkyl and hydroxy;
Ar is selected from the group consisting of: phenyl, pyrimidin-2-yl, pyrimidin-4-yl and pyridin-3-yl;
n is 0, 1 or 2;
x is 0, 1, 2 or 3; and
m is 0 or 1.

14. A process for the preparation of a compound of formula I 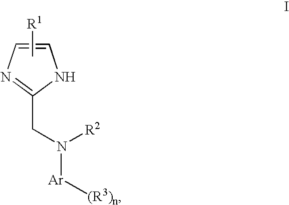
said process comprising removing a protecting group of compounds of formulas V or VIII 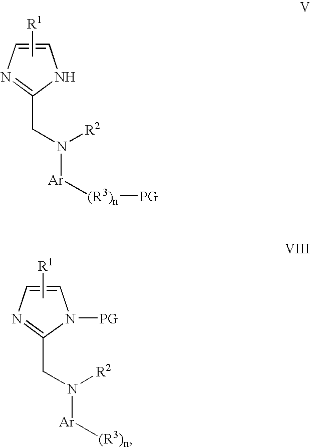
wherein, in the above formulas,
R¹ is selected from the group consisting of hydrogen and lower alkyl;
R² is selected from the group consisting of: hydrogen, lower alkyl, lower alkenyl, lower alkyl substituted by hydroxy,
lower alkyl substituted by halogen,
—(CH₂)ₓ—S-lower alkyl,
—(CH₂)ₓ—O-lower alkyl,
—(CH₂)ₓ—NHC(O)O-lower alkyl,
—(CH₂)ₓ-aryl and
—(CH₂)ₓ-heteroaryl;
each R³ is independently selected from the group consisting of: hydrogen, lower alkyl, lower alkoxy, halogen, hydroxy, lower alkyl substituted by halogen,
—O—(CH₂)ₘ-aryl,
—O—(CH₂)ₘ-heteroaryl,
—(CR₂)ₘ-aryl and
—(CR₂)ₘ-heteroaryl;
each R is independently selected from the group consisting of: hydrogen, lower alkyl and hydroxy;
Ar is selected from the group consisting of: phenyl, pyrimidin-2-yl, pyrimidin-4-yl and pyridin-3-yl;
n is 0, 1 or 2;
x is 0, 1, 2 or 3;
m is 0 or 1; and
PG is a protecting group.

15. A process for the preparation of a compound of formula I 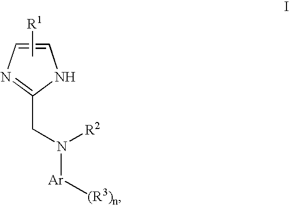
said process comprising reducing a compound of formula VII 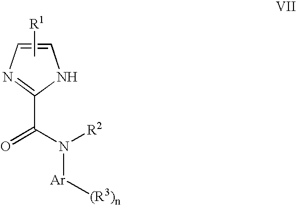
to form a compound of formula VIII 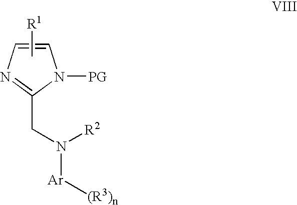
and removing the protecting group,
wherein, in the above formulas,
R¹ is selected from the group consisting of hydrogen and lower alkyl;
R² is selected from the group consisting of: hydrogen, lower alkyl, lower alkenyl, lower alkyl substituted by hydroxy,
lower alkyl substituted by halogen,
—(CH₂)ₓ—S-lower alkyl,
—(CH₂)ₓ—O-lower alkyl,
—(CH₂)ₓ—NHC(O)O-lower alkyl,
—(CH₂)ₓ-aryl and
—(CH₂)ₓ-heteroaryl;
each R³ is independently selected from the group consisting of: hydrogen, lower alkyl, lower alkoxy, halogen, hydroxy, lower alkyl substituted by halogen,
—O—(CH₂)ₘ-aryl,
—O—(CH₂)ₘ-heteroaryl,
—(CR₂)ₘ-aryl and
—(CR₂)ₘ-heteroaryl;
each R is independently selected from the group consisting of: hydrogen, lower alkyl and hydroxy;
Ar is selected from the group consisting of: phenyl, pyrimidin-2-yl, pyrimidin-4-yl and pyridin-3-yl;
n is 0, 1 or 2;
x is 0, 1, 2 or 3;
m is 0 or 1; and
PG is a protecting group.

16. A composition comprising a compound according to claim 1 selected from the group consisting of:
(1H-imidazol-2-ylmethyl)-phenyl-amine,
(4-fluoro-phenyl)-(1H-imidazol-2-ylmethyl)-amine,
(4-chloro-phenyl)-(1H-imidazol-2-ylmethyl)-amine,
(1H-imidazol-2-ylmethyl)-(2-methoxy-phenyl)-amine,
(3-chloro-phenyl)-(1H-imidazol-2-ylmethyl)-amine,
benzyl-(1H-imidazol-2-ylmethyl)-phenyl-amine,
ethyl-(1H-imidazol-2-ylmethyl)-phenyl-amine, and
(3,4-dichloro-phenyl)-(1H-imidazol-2-ylmethyl)-amine; and
a pharmaceutically acceptable excipient.

17. A composition according to claim 16 for use in the treatment of depression, anxiety disorders, bipolar disorder, attention deficit hyperactivity disorder, stress-related disorders, psychotic disorders, schizophrenia, neurological diseases, Parkinson's disease, neurodegenerative disorders, Alzheimer's disease, epilepsy, migraine, hypertension, substance abuse and metabolic disorders, eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders.

18. A composition according to claim 17 for use in the treatment of depression, psychosis, Parkinson's disease, anxiety and attention deficit hyperactivity disorder (ADHD).
